(19) United States
(12) Patent Application Publication
(10) Pub. No.: US 2005/0142574 A1
(43) Pub. Date: Jun. 30, 2005

(54) Mutants of core 2 B-1, 6-N-acetylglycosaminyltransferase

(75) Inventors: Steve Bryson, Toronto (CA); Dale Cumming, Acton, MA (US); Alessandro Datti, Toronto (CA); James Rini, Toronto (CA); Sawako Takeshita, Tokyo (JP); Roderick Tse, Willowdale (CA); April Lew, Toronto (CA)

Correspondence Address: QUINE INTELLECTUAL PROPERTY LAW GROUP, P.C., P O BOX 458, ALAMEDA, CA, 94501, US

(73) Assignee: Seikagaku Corporation and Glycodesign Holdings Ltd.

(21) Appl. No.: 10/866,526

(22) Filed: Jun. 10, 2004

Related U.S. Application Data
(60) Provisional application No. 60/477,649, filed on Jun. 10, 2003.

(51) Int. Cl. C12Q 1/68; C07H 21/04; C08B 37/00; C12P 19/28; C12N 9/10

(52) U.S. Cl. 435/6; 435/69.1; 435/85; 435/193; 435/320.1; 435/325; 536/23.2; 536/53

(57) ABSTRACT

This invention provides novel mutant Core 2 GlcNAcT nucleic acids, polypeptides encoded by the nucleic acids, and uses of the nucleic acids and polypeptides

### CROSS-REFERENCE TO RELATED APPLICATIONS

This application claims benefit of and priority to U.S. Ser. No. 60/477,649, filed on Jun. 10, 2003 which is incorporated herein by reference in its entirety for all purposes.

### STATEMENT AS TO RIGHTS TO INVENTIONS MADE UNDER FEDERALLY SPONSORED RESEARCH AND DEVELOPMENT

[Not Applicable]

### FIELD OF THE INVENTION

The invention relates to mutant polypeptides of Core 2β-1,6-N-acetylglycosaminyltransferase (Core 2 GlcNAcT) or portions thereof, as well as nucleic acid molecules encoding such polypeptides, and processes for producing such polypeptides using recombinant techniques.

### BACKGROUND OF THE INVENTION

The enzyme UDP-GlcNAc:Gal[β] 1,3GalNAc-R (GlcNAc to GalNAc) [β]1,6-N-acetylglucosaminyltransferase (i.e. Core 2 β-1,6-N-acetylglycosaminyltransferase, Core 2 GlcNAcT) converts core 1 (i.e. Gal[β]1,3GalNAc[α]-O) to Core 2 structures (i.e. Gal[β] 1,3[GlcNAc[β] 1,6]GalNAc[α]-O in the O-linked glycan biosynthesis pathway (Williams and Schachter, J. Biol. Chem 255: 11247, 1980 and Schachter H. and Brockhausen, I, In: Allen, H. J. and Kisailus, E. C. (eds) Glycoconjugates. Composition, Structure, and Function. Marcel Dekker, New York, pp 263-332). Core 2 GlcNAcT activity is important in the extension of O-linked sugars with poly(N-acetyllactosamine) (i.e. repeating Gal [β]1-4GlcNAc [β] 1-3). These structures have been associated with malignant transformation (Yousefi et al. (1991) J. Biol. Chem. 266(3): 1772-1782.) and proliferative activation of lymphocytes (Higgins et al. (1991) J. Biol. Chem., 266(10): 6280-6290), they affect cellular adhesion (Zhu and Laine (1985) J. Biol. Chem., 260(7):4041-4045; Laferte and Dennis (1988) Cancer Res.; 48(17): 4743-4748), and they may act as ligands for mammalian lectins (Merkle and Cummings (1988) J. Biol. Chem. 263(31): 16143-9).

Core 2 GlcNAcT is a key enzyme in the modulation of cell-cell interactions through glycosylation of target molecules. For example, glycosylation of P-selectin glycoprotein ligand (“PSGL-1”) modulated by Core 2 GlcNAcT has been found to be a critical step for binding to P-selectin (Kumar et al. (1996) Blood, 88(10): 3872-3879.; Li et al. (1996) J. Biol. Chem. 271(6): 3255-3264.). Diabetes and hyperglycemia induce Core 2 GlcNAcT gene expression specifically in cardiac myocytes of rats, and expression of Core 2 GlcNAcT in diabetic heart has also been associated with a stress-response and myocardial hypertrophy (Nishio et al., (1995) J. Clin Invest., 96(4): 1759-1767).

A somatic mutation that prevents UDP-Gal transport into the Golgi, blocking O- and N-linked extensions including Core 2 structures, causes a more severe attenuation of metastasis than a block in either pathway alone, suggesting both O-linked Core 2 and N-linked branched oligosaccharides contribute to the malignant phenotype. In this regard, it was shown that GalNAcαR prevents Core 2 synthesis by blocking one enzyme earlier in the O-linked pathway, and it reduces invasion and metastasis. It has also been found that an increased expression of Core 2 GlcNAcT in colorectal cancer cells is closely correlated with the progression of the disease (Shimodaira et al. (1997) Cancer Res., 57(23): 5201-5206).

The citation of any reference herein is not an admission that such reference is available as prior art to the instant invention.

### SUMMARY OF THE INVENTION

The present invention provides novel mutant polypeptides of Core 2 β-1,6-N-acetylglycosaminyltransferase (Core 2 GlcNAcT). A Core 2 GlcNAcT mutant polypeptide, (and/or in the appropriate context a fusion or chimeric Core 2 GlcNAcT mutant polypeptide), is sometimes referred to herein as “C2 mutant” or “C2 mutant polypeptide”.

In an aspect of the invention, an isolated Core 2 GlcNAcT polypeptide is provided wherein amino acid 217 is any amino acid other than cysteine.

A particular mutant of the present invention is a polypeptide having an amino acid sequence of a Core 2 GlcNAcT wherein amino acid residues of the Core 2 GlcNAcT are replaced or deleted providing a Core 2 GlcNAcT with increased stability while retaining Core 2 GlcNAcT activity.

In an aspect the original cysteine residue corresponding to position 217 of a naturally occurring Core 2 GlcNAcT is replaced by a substitution amino acid residue or is deleted. Alteration of cysteine 217 of Core 2 GlcNAcT increases stability of the enzyme while retaining Core 2 GlcNAcT activity.

In a particularly preferred embodiment, the cysteine residue at original amino acid position 217 is replaced by a serine residue or alanine residue.

Core 2 GlcNAcT mutant polypeptides of the present invention have Core 2 GlcNAcT activity and have enhanced stability. In certain embodiments, Core 2 GlcNAcT mutant polypeptides of the invention also have Kₘ values for the Core 2 GlcNAcT1 donor substrate and acceptor substrate that can be substantially the same as a native Core 2 GlcNAcT1. The inhibitory potential of the glycosyltransferase products, UDP and GlcNAc(β1-6)Gal(β1-3)GalNAcα-pNp, can be substantially the same for a C2 mutant of the invention and a native Core 2 GlcNAcT1. Similar to the native or wild type enzyme, C2 mutants of the invention do not use UDP-Glc as a substrate. In certain embodiments, this invention contemplates mutant polypeptides that comprise conservative substitutions of the mutant polypeptides described herein.

Other additions, substitutions, and/or deletions may be made to the C2 mutants of the present invention. For example, a mouse C2 mutant may optionally include a second amino acid substitution at position cysteine 235. Alternatively, one or more of the terminal amino acid residues may be deleted from the nucleic acid sequence, as is known to those skilled in the art, while substantially retaining the improved properties of the C2 mutants. In an embodiment, N-terminal amino acid residues are deleted.

The present invention also relates to nucleic acid molecules or polynucleotides encoding a C2 mutant polypeptide.

The polynucleotides can be used to transform host cells to express the C2 mutants of the invention. They can also be used as a probe to detect related enzymes. The polynucleotides can be used as DNA sizing standards.

The present invention still further relates to recombinant vectors that include the nucleic acid molecules of the invention. The nucleic acid molecules of the invention may be inserted into an appropriate vector, and the vector may contain the necessary elements for the transcription and translation of an inserted coding sequence. Accordingly, vectors may be constructed which comprise a nucleic acid molecule of the invention, and where appropriate one or more transcription and translation elements linked to the nucleic acid molecule.

A vector can be used to transform host cells. Therefore, the invention provides host cells containing a vector of the invention. As well, the invention provides methods of making such vectors and host cells.

The mutant C2 polypeptides of the invention can be encoded, expressed, and purified by any one of a number of methods known to those skilled in the art. Preferred production methods will depend on many factors including the costs and availability of materials and other economic considerations. The optimum production procedure for a given situation will be apparent to those skilled in the art through minimal experimentation.

In accordance with an aspect of the present invention, there is provided a process for producing a C2 mutant by recombinant techniques utilizing the nucleic acid molecules of the invention. The method may comprise culturing recombinant host cells containing a nucleic acid sequence encoding a C2 mutant, under conditions promoting expression of the C2 mutant, and subsequent recovery of the C2 mutant.

In one aspect the invention provides a method for preparing a C2 mutant comprising: (a) transferring a vector of the invention comprising a nucleic acid molecule encoding a C2 mutant into a host cell; (b) selecting transformed host cells from untransformed host cells; (c) culturing a selected transformed host cell under conditions which allow expression of the C2 mutant; and (d) isolating the C2 mutant.

The invention further contemplates a recombinant C2 mutant polypeptide obtained using a method of the invention.

A C2 mutant polypeptide of the invention may be conjugated with other molecules, such as polypeptides, to prepare fusion polypeptides or chimeric polypeptides. This may be accomplished, for example, by the synthesis of N-terminal or C-terminal fusion polypeptides.

The invention further contemplates antibodies having specificity against a C2 mutant polypeptide of the invention. Antibodies may be labeled with a detectable substance and used to detect C2 mutant polypeptides. In another embodiment, the invention provides an isolated antibody that binds specifically to a C2 mutant polypeptide.

Because of their improved properties, the C2 mutants of the present invention are particularly well suited for use in screening methods for identifying modulators of Core 2 GlcNAcTs. In one aspect of the invention, a method is provided wherein the C2 mutants are used to identify inhibitors of Core 2 GlcNAcTs.

Still further the invention provides a method for evaluating a test compound for its ability to modulate the biological activity of Core 2 GlcNAcTs. “Modulate” refers to a change or an alteration in the biological activity of a Core 2 GlcNAcT. Modulation may be an increase or a decrease in activity, a change in characteristics (e.g., kinetic characteristics), or any other change in the biological, functional, or immunological properties of the polypeptide.

In an embodiment of the invention a method is provided for screening a compound for effectiveness as an antagonist of a Core 2 GlcNAcT, comprising the steps of a) contacting a C2 mutant polypeptide with a test compound, under conditions wherein antagonist activity of the polypeptide can be detected, and b) detecting antagonist activity.

The substances and compounds identified using the methods of the invention, may be used to modulate the biological activity of a Core 2 GlcNAcT, and they may be used in the treatment of conditions mediated by a Core 2 GlcNAcT such as inflammatory disorders, liver disorders, kidney disorders, skeletal muscle disorders, cardiovascular disorders, diabetes, infectious diseases, hormonal disorders, parasitic diseases, and proliferative diseases such as cancer. Accordingly, the substances and compounds may be formulated into compositions for administration to individuals suffering from one or more of these conditions. Therefore, the present invention also relates to a composition comprising one or more of a substance or compound identified using a method of the invention, and a pharmaceutically acceptable carrier, excipient or diluent. A method for treating or preventing these conditions is also provided comprising administering to a patient in need thereof, a composition of the invention.

Having provided a novel C2 mutant polypeptide with Core 2 transferase activity, and nucleic acids encoding same, the invention accordingly further provides methods for preparing oligosaccharides and/or polysaccharides e.g., two or more saccharides, including sLe^{x} antigens. In specific embodiments, the invention relates to a method for preparing an oligosaccharide comprising contacting a reaction mixture comprising a sugar donor, and an acceptor in the presence of a C2 mutant polypeptide of the invention.

In accordance with a further aspect of the invention, there are provided processes for utilizing polypeptides or nucleic acid molecules, for in vitro purposes related to scientific research, synthesis of DNA and manufacture of vectors.

These and other aspects, features, and advantages of the present invention should be apparent to those skilled in the art from the following drawings and detailed description.

### BRIEF DESCRIPTION OF THE DRAWINGS

The invention will be better understood with reference to the drawings in which:

FIG. 1 is a graph illustrating that the C2 mutant mouse C217S is not inhibited by the reagents that target free thiol groups.

FIG. 2 is a graph illustrating that the C2 mutant human C217S is not inhibited by the reagents that target free thiol groups.

FIGS. 3A and 3B provide an analysis of the small scale purification of FLAG-tagged wild type mouse Core 2 GlcNAcT1 and FLAG-tagged C217S mouse Core 2 GlcNAcT1 by SDS-PAGE with SimplyBlue™ SafeStain (FIG. 3A) and by Western blotting with an anti-FLAG antibody (FIG. 3B). FIG. 3A: lane 1: M.W. marker (SeeBlue Plus2™); lane 2:20 μL of culture supernatant from the cells transfected with wild-type mouseCore2L-FLAG construct; lane 3:3 μg of purified wild mouseCore2L-FLAG polypeptide; lane 4:20 μL of culture supernatant from the cells transfected with C217S mutant mouseCore2L-FLAG construct; lane 5:3 μg of purified C217S mutant mouseCore2L-FLAG polypeptide. FIG. 3B: lane 1: M.W. marker (SeeBlue Plus2™); lane 2:10 μL of culture supernatant from the cells transfected with wild-type mouseCore2L-FLAG construct; lane 3:0.1 μL of purified wild mouseCore2L-FLAG polypeptide; lane 4:10 μL of culture supernatant from the cells transfected with C217S mutant mouseCore2L-FLAG construct; lane 5:0.1 μg of purified C217S mutant mouseCore2L-FLAG polypeptide.

FIGS. 4A and 4B provide an analysis of the large scale purification of FLAG-tagged C217S mouse Core 2 GlcNAcT1 by SDS-PAGE with SimplyBlue™ SafeStain (FIG. 4A) and with Silver Stain Plus™ (FIG. 4B). FIG. 4A lane 1: M.W. marker (SeeBlu Plus2™), lane 2:2 microgram purified mouse C217S mutant polypeptide. FIG. 4B lane 1: M.W. marker (SeeBlu Plus2™), lane 2:2 microgram purified mouse C217S mutant polypeptide.

### BRIEF DESCRIPTION OF THE SEQUENCES

SEQ ID NO:1 is a nucleic acid sequence encoding human Core 2 GlcNAcT1. (GenBank Accession No. M97347.)

SEQ ID NO:2 is a nucleic acid sequence encoding human Core 2 GlcNAcT1 fusion construct.

SEQ ID NO:3 is an amino acid sequence of human Core 2 GlcNAcT1. (GenBank Accession No. AAA35919.)

SEQ ID NO:4 is an amino acid sequence of human Core 2 GlcNAcT1 fusion construct.

SEQ ID NO:5 is a nucleic acid sequence encoding mouse Core 2 GlcNAcT1. (GenBank Accession No. U19265.)

SEQ ID NO:6 is a nucleic acid sequence encoding mouse Core 2 GlcNAcT1 fusion construct.

SEQ ID NO:7 is an amino acid sequence of mouse Core 2 GlcNAcT1. (GenBank Accession No. Q09324.)

SEQ ID NO:8 is an amino acid sequence of mouse Core 2 GlcNAcT1 fusion construct.

SEQ ID NO:9 is an amino acid sequence of a human Core 2 GlcNAcT1 Cys217→Ser mutant polypeptide.

SEQ ID NO:10 is an amino acid sequence of a human Core 2 GlcNAcT1/S-tag fusion protein including Tag and EK site in bold and Cys217→Ser mutation in large print.

SEQ ID NO:11 is an amino acid sequence of a mouse Core 2 GlcNAcT1 Cys217→Ser mutant polypeptide.

SEQ ID NO:12 is an amino acid sequence of a mouse Core 2 GlcNAcT1 Cys217→Ser mutation/FLAG-tag fusion protein including Tag and EK site in bold and Cys217→Ser mutation in large print.

SEQ ID NO:13 is a nucleic acid sequence encoding a human Core 2 GlcNAcT1 Cys217 →Ser mutant polypeptide.

SEQ ID NO:14 is a nucleic acid sequence encoding a human Core 2 GlcNAcT1 Cys217→Ser mutation construct.

SEQ ID NO:15 is a nucleic acid sequence encoding a mouse Core 2 GlcNAcT1 Cys217→Ser mutant polypeptide.

SEQ ID NO:16 is a nucleic acid sequence encoding a mouse Core 2 GlcNAcT1 Cys217→Ser mutation construct.

SEQ ID NO:17 is an amino acid sequence of a human Core 2 GlcNAcT1 Cys217→Ala mutant polypeptide.

SEQ ID NO:18 is an amino acid sequence of a human Core 2 GlcNAcT1 Cys217 →Ala mutation construct.

SEQ ID NO:19 is an amino acid sequence of a mouse Core 2 GlcNAcT1 Cys217→Ala mutant polypeptide.

SEQ ID NO:20 is an amino acid sequence of a mouse Core 2 GlcNAcT1 Cys217→Ala mutation construct.

SEQ ID NO:21 is a nucleic acid sequence encoding a human Core 2 GlcNAcT1 Cys217→Ala mutant polypeptide.

SEQ ID NO:22 is a nucleic acid sequence encoding a human Core 2 GlcNAcT1 Cys217→Ala mutation construct.

SEQ ID NO:23 is a nucleic acid sequence of a mouse Core 2 GlcNAcT1 Cys217→Ala mutant polypeptide.

SEQ ID NO:24 is a nucleic acid sequence encoding a mouse Core 2 GlcNAcT1 Cys217 →Ala mutation construct.

SEQ ID NO:25 is an amino acid sequence identified in a mutant Core 2 GlcNAcT1 polypeptide of the invention.

SEQ ID NO:26 is a mutagenic primer used in the preparation of a mouse Cys 217S mutant polypeptide.

SEQ ID NO:27 is a mutagenic primer used in the preparation of a mouse Cys 217T mutant polypeptide.

SEQ ID NO:28 is a mutagenic primer used in the preparation of a mouse Cys 217A mutant polypeptide.

SEQ ID NO:29 is a nucleic acid sequence encoding a human Core 2 GlcNAcT1 wildtype construct.

SEQ ID NO:30 is a nucleic acid sequence encoding a mouse Core 2 GlcNAcT1 wildtype construct.

### DETAILED DESCRIPTION

In accordance with the present invention there may be employed conventional molecular biology, microbiology, and recombinant DNA techniques within the skill of the art. Such techniques are explained fully in the literature. See for example, Sambrook, Fritsch, & Maniatis, Molecular Cloning: A Laboratory Manual, Second Edition (1989) Cold Spring Harbor Laboratory Press, Cold Spring Harbor, N.Y); DNA Cloning: A Practical Approach, Volumes I and II (D. N. Glover ed. 1985); Oligonucleotide Synthesis (M. J. Gait ed. 1984); Nucleic Acid Hybridization B. D. Hames & S. J. Higgins eds. (1985); Transcription and Translation B. D. Hames & S. J. Higgins eds (1984); Animal Cell Culture R. I. Freshney, ed. (1986); Immobilized Cells and enzymes IRL Press, (1986); and B. Perbal, A Practical Guide to Molecular Cloning (1984).

Glossary

The term “Core 2 GlcNAcT” or “Core 2 transferase”, as used herein, includes naturally occurring (“native” or “wildtype”) Core 2 GlcNAcTs, as well as non-naturally occurring polypeptides (e.g., recombinant polypeptides) having an amino acid sequence duplicative of that of naturally occurring Core 2 GlcNAcT which catalyses a reaction identical to that of native protein when expressed in a host cell. The term also includes allelic, species and tissue variants. An allelic variant differs from a native or wild type Core 2 GlcNAcT by only one, or at most, a few amino acid substitutions. A species variation of the polypeptide is a variation that is naturally occurring among different species of an organism. A Core 2 GlcNAcT may be a Core 2 GlcNAcT L form (T1), in particular, human Core 2 GlcNAcT [see SEQ ID NO:3; see also GenBank Accession No. AAA35919(aa) and M97347(nt)], mouse Core 2 GlcNAcT [see SEQ ID NO:7; see also GenBank Accession No. Q09324 (aa) and U19265 (nt)], or a Core 2 GlcNAcT from another species.

“Core 2 GlcNAcT activity” or “Core 2 transferase activity” refers to the conversion of core 1 to Core 2 structures by a Core 2 GlcNAcT. Catalytic activity of a Core 2 GlcNAcT is substantially the same as a native Core 2 GlcNAcT. Catalytic activity is considered “substantially the same” if it is identical to the activity obtained for a native Core 2 GlcNAcT, or it may be 2 to 10 fold, 2 to 5 fold, or 2 to 3 fold less active compared to the activity of a native Core 2 GlcNAcT.

Catalytic activity is assessed by comparing the activity of a C2 mutant (e.g., catalytic efficiency and kinetic parameters) with that of a wild type or native Core 2 GlcNAcT. Activity of a C2 mutant may be measured against one or more test substrates, for example, as illustrated in the examples.

The term “mutant” as used herein refers to a Core 2 GlcNAcT where at least one of the amino acid residues is replaced by a substitution amino acid residue, or is deleted. A mutant polypeptide has advantageous properties compared to the wildtype polypeptide. In an aspect, the mutant polypeptide has increased stability compared to the wildtype polypeptide.

The term “substitution amino acid” refers to an amino acid which replaces a naturally occurring amino acid, and which is different from the original amino acid. A substitution amino acid is selected so that it does not block critical substrate interactions or drastically alter folding/conformation of the enzyme. In certain embodiments, a substitution amino acid imparts increased stability to the mutant and is generally an amino acid that is not sensitive to oxidation, alkylation, and/or heavy metal binding. In particular, a substitution amino acid is insensitive to inactivation caused by air oxidation, oxidative agents, thiol-reactive agents, and heavy metals. Examples of such amino acids include the natural amino acids serine (Ser), alanine (Ala), and valine (Val), and corresponding unnatural amino acids in their D and L stereoisomers (e.g., N-methylvaline, norvaline), and their analogs.

The term “analogs” in reference to an amino acid refers to an amino acid wherein either the C-terminal carboxy group, the N-terminal amino group or side-chain functional group has been chemically modified to another functional group. Amino acid analogs include the natural and unnatural amino acids which are chemically blocked, reversibly or irreversibly, or modified on their N-terminal amino group or their side-chain groups. Examples of amino acid analogs of serine, alanine, and valine include but are not limited to L/D-N-methylserine, L/D-N-methyl alanine, L/D-N-methylvaline, L-serine hydroxamate, N-(2-aminoethyl)-b-alanine, D-valine-OH, L-alanine-OH, L/D-serine(tBu)—OH, N-Methyl-L/D-alanine-OH, and N-methyl-L/D-valine-OH.

In an embodiment of the invention, the substitution amino acid is serine at position cysteine 217 of a wildtype Core 2 GlcNAcT.

In another embodiment of the invention, the substitution amino acid is alanine at position cysteine 217 of a wildtype Core 2 GlcNAcT.

“Stability” as used herein in relation to a C2 mutant polypeptide means the C2 mutant polypeptide is not sensitive, or has decreased sensitivity to oxidation, alkylation, or heavy metal binding. In particular, it refers to a decreased sensitivity to thiol targeted reagents compared to the wildtype enzyme. In an aspect, the term refers to insensitivity to inactivation caused by air oxidation, oxidative agents, thiol-reactive agents, and heavy metals.

The terms “nucleic acid” or “nucleic acid molecule” or “oligonucleotide” or grammatical equivalents herein refer to at least two nucleotides covalently linked together. These terms are intended to include modified or unmodified DNA, RNA, or a mixed polymer, and can be either single-stranded, double-stranded or triple-stranded, and represents the sense or antisense strand. For example, a nucleic acid sequence may be a single-stranded or double-stranded DNA, DNA that is a mixture of single- and double-stranded regions, or single-, double- and triple-stranded regions, single- and double-stranded RNA, RNA that may be single-stranded, or more typically, double-stranded, or triple-stranded, or a mixture of regions comprising RNA or DNA, or both RNA and DNA. The strands in such regions may be from the same molecule or from different molecules. The DNAs or RNAs may contain one or more modified bases. For example, the DNAs or RNAs may have backbones modified for stability or for other reasons. Thus, nucleic acid analogs are included that may have alternate backbones, comprising, for example, phosphoramide (Beaucage et al. (1993) Tetrahedron 49(10): 1925) and references therein; Letsinger (1970) J. Org. Chem. 35: 3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 1419), phosphorothioate (Mag et al. (1991) Nucleic Acids Res. 19: 1437; and U.S. Pat. No. 5,644,048), phosphorodithioate (Briu et al. (1989) J. Am. Chem. Soc. 111 : 2321, O-methylphophoroamidite linkages (see Eckstein, Oligonucleotides and Analogues: A Practical Approach, Oxford University Press), and peptide nucleic acid backbones and linkages (see Egholm (1992) J. Am. Chem. Soc. 114: 1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Letsinger et al. (1988) J. Am. Chem. Soc. 110: 4470; Letsinger et al. (1994) Nucleoside & Nucleotide 13: 1597; Chapters 2 and 3, ACS Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR 34: 17; Tetrahedron Lett. 37: 743 (1996)) and non-ribose backbones, including those described in U.S. Pat. Nos. 5,235,033 and 5,034,506, and Chapters 6 and 7, ACS Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp 169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

The term “nucleic acid molecule” and in particular DNA or RNA, refers only to the primary and secondary structure and it does not limit it to any particular tertiary forms.

The terms “amino acid” or “amino acid residue” as used herein refers to natural, synthetic, or modified amino acids.

The terms “polypeptide”, “peptide” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers.

The term “conservative substitution” is used in reference to proteins or peptides to reflect amino acid substitutions that do not substantially alter the activity and/or binding affinity of the molecule. Typically conservative amino acid substitutions involve substitution one amino acid for another amino acid with similar chemical properties (e.g., charge or hydrophobicity). The following six groups each contain amino acids that are typical conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

A “fusion molecule” refers to any molecule that is to be attached (directly or though a linker) to a Core 2 GlcNAcT polypeptide of this invention. Preferred fusion molecules are proteins that can be expressed as a fusion protein with the Core 2 GlcNAcT polypeptide.

The term “specifically binds”, as used herein, when referring to a biomolecule (e.g., protein, nucleic acid, antibody, etc.), refers to a binding reaction which is determinative of the presence of a biomolecule in a heterogeneous population of molecules (e.g., proteins and other biologics). Thus, under designated conditions (e.g., immunoassay conditions in the case of an antibody or stringent hybridization conditions in the case of a nucleic acid), the specified ligand or antibody binds to its particular “target” molecule and does not bind in a significant amount to other molecules present in the sample. In certain embodiments, an antibody specifically binds its target (e.g., a Core 2 GlcNAcT polypeptide of this invention) with a Kd greater than about 10⁻⁶ M or 10⁻⁷ M, preferably greater than about 10⁻⁸ M, still preferably greater than about 10⁻⁹ M, and most preferably greater than about 10⁻¹⁰ M.

Nucleic Acid Molecules of the Invention

As hereinbefore mentioned, in certain embodiments, the invention provides nucleic acid molecules encoding C2 mutant polypeptides.

In one embodiment, of the invention an isolated nucleic acid molecule is contemplated which comprises:
(i) a nucleic acid sequence encoding a C2 mutant polypeptide comprising or consisting essentially of SEQ. ID. NO. 9, 10, 11, 12, 17, 18, 19, and 20;
(ii) a nucleic acid sequence complementary to (i);
(iii) a nucleic acid sequence differing from any of (i) or (ii) in codon sequences due to the degeneracy of the genetic code; and/or
(iv) an allelic or species variation of (i), (ii) or (iii).

In a specific embodiment, the isolated nucleic acid molecule comprises:
(i) a nucleic acid sequence comprising or consisting essentially of SEQ. ID. NO. 13, 14, 15, 16, 21, 22, 23, and 24;
(ii) nucleic acid sequences complementary to (i);
(iii) nucleic acid sequences differing from any of the nucleic acid sequences of (i) or (ii) in codon sequences due to the degeneracy of the genetic code; and/or
(iv) an allelic or species variation of (i), (ii) or (iii).

The term “complementary” refers to the natural binding of nucleic acid molecules under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between the single stranded molecules.

In a preferred embodiment an isolated nucleic acid molecule of the invention comprises a nucleic acid sequence encoded by the amino acid sequence shown in SEQ. ID. NO. 9, 10, 11, 12, 17, 18, 19, and 20; or comprises the nucleic acid sequence shown in SEQ. ID. NO. 13, 14, 15, 16, 21, 22, 23, and 24 wherein T can also be U.

Isolated nucleic acid molecules encoding a C2 mutant polypeptide and comprising a sequence that differs from the nucleic acid sequence of SEQ. ID. NO. 9, 10, 11, 12, 17, 18, 19, or 20 due to degeneracy in the genetic code are also within the scope of the invention. Such nucleic acids encode equivalent polypeptides but differ in sequence from the sequence of SEQ. ID. NO. 13, 14, 15, 16, 21, 22, 23, and 24, due to degeneracy in the genetic code. For example, species variations i.e. variations in nucleotide sequence naturally occurring among different species, are contemplated.

Mutations may be introduced into Core 2 GlcNAcT nucleic acid molecules using methods well known to those of ordinary skill in the art. In an embodiment, C2 mutants are prepared by site-directed mutagenesis of a DNA encoding a Core 2 GlcNAcT. Techniques for performing site-directed mutagenesis or non-random mutagenesis are known in the art and include but are not limited to oligonucleotide-mediated mutagenesis (Adellman et al., (1983) DNA, 2: 183), cassette mutagenesis (Wells et al. (1985) Gene 344: 315), and binding mutagenesis (Ladner et al WO88/06630). Protocols for site-directed modification of proteins are well known in the art (see, for example, U.S. Pat. Nos. 5,932,419, 5,789,166, 5,705,479, 5,635,475, 5,556,747, 5,354,670, 5,352,779, 5,284,760, and 5,071,743). In addition, kits for site-directed mutagenesis are commercially available (see TRANSFORMER™, Site-Directed Mutagenesis kit available from BD Biosciences Clontech; MutaGene phagemid in vitro mutagenesis kit available from Bio-Rad; and the QUICKCHANGE™ Site Directed Mutagenesis kit from Stratagene®).

In one embodiment, the substitute amino acid residue (e.g., serine, or, in certain embodiments, any amino acid other than cysteine or serine) is introduced into the selected position (e.g., cysteine 217) by oligonucleotide-mediated mutagenesis using the polymerase chain reaction technique. A nucleic acid molecule encoding a Core 2 GlcNAcT is carried by a suitable plasmid which can be chosen by persons skilled in the art for convenience or as desired. Examples of plasmids include plasmids from the pBR, pUC, pUB, pET, pTRiEx, pFLAG, or pHY4 series.

For site-directed mutagenesis, a fragment containing a selected mutation site may be cleaved by restriction endonucleases from a gene encoding a native Core 2 GlcNAcT. The fragment is used as a template in a modified PCR technique (Higushi et al. (1988) Nucleic Acid Res., 16: 7351-7367). An oligonucleotide containing the desired mutation is used as a mismatch primer to initiate chain extension between 5′ and 3′ PCR flanking primers. In general, the procedure involves generating a DNA fragment containing the desired base substitution (e.g., cys 217 to ser) using the mismatch primer and the 5′ primer. The DNA fragment is separated from the primers by electrophoresis, and used as the new 5′ primer in a second PCR reaction with the 3′ primer to generate the complete fragment containing the desired base substitution. The mutation is confirmed by sequencing and inserted back to the position of the original fragment.

In a particular embodiment of the invention, a C2 mutant is produced using the site-directed mutagenesis technique described in U.S. Pat. Nos. 5,789,166, 5,932,419, and 6,391,548 to Bauer et al.

More than one mutation can be introduced into a Core 2 GlcNAcT, preferably retaining the properties of the C2 mutant described herein. For example, mutations (e.g., additions, substitutions, and/or deletions) can be introduced at positions in addition to cysteine 217, and mutations can create new restriction sites, change codon preference, or produce splice variants. In an embodiment, a mouse C2 mutant may optionally include a second amino acid substitution at cysteine 235. In another embodiment, N-terminal amino acids are deleted.

Nucleic acid molecules of the invention may be chemically synthesized using standard techniques. Methods of chemically synthesizing polydeoxynucleotides are known, including but not limited to solid-phase synthesis, which like peptide synthesis, has been fully automated in commercially available DNA synthesizers (see e.g., Itakura et al. U.S. Pat. No. 4,598,049; Caruthers et al. U.S. Pat. No. 4,458,066; and Itakura U.S. Pat. Nos. 4,401,796 and 4,373,071).

C2 Mutant Polypeptides

In an aspect, the amino acid sequence of an isolated Core 2 mutant polypeptide of the invention comprises the sequence of a Core 2 GlcNAcT with an amino acid substitution or deletion that results in increased stability compared to the wild type Core 2 GlcNAcT while retaining the catalytic activity of a Core 2 GlcNAcT.

In one aspect the original cysteine residue corresponding to position 217 of a naturally occurring Core 2 GlcNAcT is replaced by a substitution amino acid residue or is deleted. It was found that alteration of cysteine 217 of Core 2 GlcNAcT increases stability of the enzyme with retention of catalytic activity.

In certain embodiments, the cysteine corresponding to position 217 of a naturally occurring Core 2 GlcNAcT polypeptide can be identified in the amino acid sequence WXYXINXCGXDFP (SEQ ID NO:25), wherein X is any amino acid residue and the cysteine is at the eighth position of the amino acid sequence. The cysteine residue is replaced or deleted as described herein. Thus, the present invention provides an isolated Core 2 GlcNAcT polypeptide comprising amino acid sequence WX¹YX² INX³X⁴GX⁵DFP (SEQ ID NO:31), wherein each of X¹, X², X³ and X⁵ is independently any amino acid residue and X⁴ is any residue other than cysteine, or, in certain embodiments, any residue other than cysteine or serine. In certain embodiments, X¹ is lysine, arginine, or histidine, X² is isoleucine, valine, or leucine, X³ is leucine, isoleucine, or valine, X⁴ is serine or alanine, and X⁵ is isoleucine, valine, or methionine.

In further embodiments, the isolated Core 2 GlcNAcT polypeptide comprises amino acid sequence WKYLINLX⁴GMDFP (SEQ ID NO:32). In particular embodiments X⁴ is serine or alanine. In particular embodiments, the naturally occurring Core 2 GlcNAcT polypeptide is a human or mouse polypeptide.

In an embodiment, the cysteine residue corresponding to original amino acid position 217 is replaced by a serine residue. In another embodiment, the cysteine residue corresponding to original amino acid position 217 is replaced by an alanine residue.

In one preferred embodiment of the invention, the C2 mutant polypeptide comprises the amino acid sequence of SEQ ID NOs:9, 11, 17, or 19.

The invention also contemplates isoforms of the polypeptides of the invention. An isoform contains the same or different number and kinds of amino acids as the polypeptide of the invention, but the isoform may have a different molecular structure. The isoforms contemplated by the present invention preferably have the same properties as a C2 mutant polypeptide (e.g., similar Core 2 function and specificity and/or insensitivity to oxidative inactivation or thiol-reactive agents). For example, an isoform may have a different glycosylation pattern compared to a polypeptide of the invention.

The present invention also includes a C2 mutant polypeptide conjugated with a selected polypeptide, or a marker polypeptide (see below), or other glycosyltransferases to produce fusion polypeptides or chimeric polypeptides.

A C2 mutant polypeptide may be prepared using recombinant DNA methods. Accordingly, the nucleic acids of the present invention having a sequence that encodes a C2 mutant polypeptide may be incorporated in a known manner into an appropriate vector which ensures good expression of the polypeptide. Possible expression vectors include but are not limited to cosmids, plasmids, phages, or modified viruses (e.g., replication defective retroviruses, adenoviruses and adeno-associated viruses), so long as the vector is compatible with the host cell used.

The invention also contemplates a vector of the invention containing a nucleic acid molecule of the invention, and the necessary regulatory sequences for the transcription and translation of the inserted polypeptide-sequence. Suitable regulatory sequences may be derived from a variety of sources, including bacterial, fungal, viral, mammalian, or insect genes (For example, see the regulatory sequences described in Goeddel (1990) Gene Expression Technology: Methods in Enzymology 185, Academic Press, San Diego, Calif.). Selection of appropriate regulatory sequences is dependent on the host cell chosen as discussed below, and may be readily accomplished by one of ordinary skill in the art. The necessary regulatory sequences may be supplied by the wildtype Core 2 GlcNAcT and/or its flanking regions.

The vectors of the invention may also contain a marker gene that facilitates the selection of host cells transformed or transfected with a recombinant molecule of the invention. Examples of marker genes are genes encoding a polypeptide such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. The markers can be introduced on a separate vector from the nucleic acid of interest.

The vectors may also contain genes that encode a fusion moiety that provides increased expression of the recombinant polypeptide; increased solubility of the recombinant polypeptide; and aid in the purification of the target recombinant polypeptide by acting as a ligand in affinity purification. For example, a proteolytic cleavage site may be added to the target recombinant polypeptide to allow separation of the recombinant polypeptide from the fusion moiety subsequent to purification of the fusion polypeptide. Typical fusion expression vectors include pGEX (Amrad Corp., Melbourne, Australia), pMAL (New England Biolabs, Beverly, Mass.), pFLAG (Sigma-Aldrich, St. Louis, Mo.), and pRIT5 (Pharmacia, Piscataway, N.J.) which fuse glutathione S-transferase (GST), maltose E binding protein, or protein A, respectively, to the recombinant polypeptide. Another expression vector system that may be utilized is the pTriEX™ system (Novagen, Madison, Wis.).

In one preferred embodiment of the invention, a C2 mutant polypeptide fusion construct comprises the amino acid sequence of SEQ ID NOs:10, 12, 18, or 20.

The vectors may be introduced into host cells to produce a transformed or transfected host cell. The terms “transfected” and “transfection” encompass the introduction of nucleic acid (e.g., a vector) into a cell by one of many standard techniques. A cell is “transformed” by a nucleic acid when the transfected nucleic acid effects a phenotypic change. Prokaryotic cells can be transfected or transformed with nucleic acid by, for example, electroporation or calcium-chloride mediated transformation. Nucleic acid can be introduced into mammalian cells via conventional techniques such as calcium phosphate or calcium chloride co-precipitation, DEAE-dextran-mediated transfection, lipofectin, electroporation or microinjection. Suitable methods for transforming and transfecting host cells can be found, for example, in Sambrook et al. (1989) Molecular Cloning: A Laboratory Manual, 2nd Edition, Cold Spring Harbor Laboratory Press, and other laboratory textbooks.

Suitable host cells include a wide variety of prokaryotic and eukaryotic host cells. For example, the polypeptides of the invention may be expressed in bacterial cells such as E. coli, insect cells (using baculovirus), yeast cells, fungal cells, insect cells, and plant or animal cells, in particular mammalian cells. Other suitable host cells can be found in Goeddel (1991) Gene Expression Technology: Methods in Enzymology 185, Academic Press, San Diego, Calif. The methods for introducing exogenous genetic material into host cells are well known to the skilled artisan.

Nucleic acid sequences coding for a C2 mutant polypeptide of the invention may include codons that are preferred for expression of the polypeptide in selected host cells, sites of cleavage by restriction endonuclease enzymes, and/or the provision of additional initial, terminal, or intermediate sequences which facilitate construction of readily expressed vectors.

A host cell may also be chosen which modulates the expression of an inserted nucleic acid sequence, or modifies, and cleaves the polypeptide in a desired fashion. Host systems or cell lines may be selected which have specific and characteristic mechanisms for post-translational processing and modification of polypeptides. For example, eukaryotic host cells including CHO, VERO, BHK, HL60, A431, HeLa, COS, MDCK, 293, 3T3, and WI38 may be used. For long-term high-yield stable expression of the polypeptide, cell lines and host systems that stably express the gene product may be engineered.

Host cells and in particular cell lines produced using the methods described herein may be particularly useful in screening and evaluating substances and compounds that modulate the activity of a Core 2 GlcNAcT.

A C2 mutant polypeptide may also be prepared by chemical synthesis using techniques well known in the chemistry of polypeptides such as solid phase synthesis (Merrifield (1964) J. Am. Chem. Assoc., 85: 2149-2154) or synthesis in homogenous solution (Houbenweyl (1987) Methods of Organic Chemistry, ed E. Wansch, Vol. 15 I and II, Thieme, Stuttgart).

N-terminal or C-terminal fusion polypeptides or chimeric polypeptides comprising a C2 mutant polypeptide of the invention conjugated with other molecules, such as polypeptides (e.g., markers or other glycosyltransferases) may be prepared by fusing, through recombinant techniques, the N-terminal or C-terminal of a C2 mutant polypeptide, and the sequence of a selected polypeptide or marker polypeptide with a desired biological function. The resultant fusion polypeptides contain a C2 mutant polypeptide fused to the selected polypeptide or marker polypeptide as described herein. Examples of peptides or polypeptides which may be used to prepare fusion polypeptides include immunoglobulins (e.g., IgG), glutathione-S-transferase (GST), protein A, HI, hemagglutinin (HA), S•tag, FLAG, β-galactosidase, maltose E binding protein, GAL, HSP, LacZ, IgG, His-tag, avidin, and truncated myc, or portions thereof.

In certain embodiments, C2 mutant polypeptides of the present invention have Core 2 GlcNAcT activity. The activity of a C2 mutant polypeptide prepared as described herein may be confirmed using methods based on HPLC (Koenderman et al. (1987) FEBS Lett. 222: 42), methods employing synthetic oligosaccharide acceptors attached to hydrophobic aglycones (Palcic et al. (1988) Glycoconjugate 5: 49; and Pierce et al. (1987) Biochem. Biophys. Res. Comm. 146: 679), or enzyme assay methods described herein.

Antibodies

A C2 mutant polypeptide of the invention can be used to prepare antibodies specific for the polypeptides. Antibodies having specificity for a C2 mutant polypeptide of the invention may also be raised from fusion polypeptides created by expressing fusion polypeptides in host cells as described herein.

Antibodies of the present invention include intact monoclonal or polyclonal antibodies, and immunologically active fragments (e.g., a Fab or (Fab)₂ fragment), an antibody heavy chain, an antibody light chain, humanized antibodies, human antibodies, a genetically engineered single chain antibody, e.g., scFv molecule, (Ladner et al., U.S. Pat. No. 4,946,778), or a chimeric antibody, for example, an antibody which contains the binding specificity of a murine antibody, but in which the remaining portions are of human origin. Antibodies, including monoclonal and polyclonal antibodies, fragments and chimeras, may be prepared using methods known to those skilled in the art.

Illustrative Applications.

The nucleic acid molecules, C2 mutant polypeptides, and antibodies of the invention may be used in methods for the identification of substances or compounds that modulate (e.g., upregulate, downregulate, etc.) the biological activity of a Core 2 GlcNAcT. The identified substances and compounds may be used for the treatment of conditions requiring modulation of a Core 2 GlcNAcT.

Methods for Identifying or Evaluating Substances/Compounds

The methods described herein are designed to identify substances and compounds that modulate the expression or biological activity of a Core 2 GlcNAcT including substances that interfere with, or enhance the expression or activity of a Core 2 GlcNAcT.

Substances and compounds identified using the methods of the invention include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies (e.g., polyclonal, monoclonal, humanized, anti-idiotypic, chimeric, single chain antibodies, fragments, e.g., Fab, F(ab)₂, and Fab expression library fragments, and epitope-binding fragments thereof), polypeptides, nucleic acids, carbohydrates, and small organic or inorganic molecules. A substance or compound can be an endogenous physiological compound or it can be a natural or synthetic compound.

A substance or compound that modulates the expression or biological activity of a Core 2 GlcNAcT can, in various embodiments, act as an agonist or antagonist. The term “agonist” refers to a molecule that increases the amount of, or prolongs the duration of, the activity of the polypeptide. The term “antagonist” refers to a molecule that decreases the amount of or duration of, the activity of the polypeptide. Agonists and antagonists may include proteins, nucleic acids, carbohydrates, small organic molecules, or any other molecules that associate with a polypeptide of the invention.

Substances that modulate a Core 2 GlcNAcT can be identified based on their ability to interfere with or enhance the activity of a C2 mutant polypeptide of the invention. The C2 mutant polypeptide of the invention may be used in any known assay system utilizing wild-type Core 2 β-1,6-N-acetylglycosaminyl transferase to screen for agonists and antagonists.

In an aspect the invention provides a method for evaluating a test substance for its ability to modulate the activity of a Core 2 GlcNAcT comprising: (a) contacting a Core 2 GlcNAcT acceptor and sugar donor with a mutant polypeptide of the invention in the presence of the test substance; and (b) comparing the amount of sugar donor transferred to acceptor in step (a) to the amount of sugar donor transferred to acceptor in the absence of the test substance.

In another aspect, the invention provides a method for evaluating a substance for its ability to modulate the activity of a Core 2 GlcNAcT comprising (a) contacting an acceptor and a sugar donor for a Core 2 GlcNAcT and a C2 mutant polypeptide of the invention in the presence of a test substance; (b) measuring the amount of sugar donor transferred to acceptor, and (c) carrying out steps (a) and (b) in the absence of the test substance to determine if the substance interferes with or enhances transfer of the sugar donor to the acceptor by the C2 mutant polypeptide.

Suitable acceptors for use in the methods of the invention include, but are not limited to saccharides, oligosaccharides, polysaccharides, glycopeptides, glycopolypeptides, or glycolipids which are either synthetic with linkers at the reducing end or naturally occurring structures, for example, an asialo-agalacto-fetuin glycopeptide and an asialo mucin. Acceptors will generally comprise β-D-galactosyl-1,3-N-acetyl-D-galactosaminyl-.

Suitable sugar donors include, but are not limited to a nucleotide sugar, dolichol-phosphate-sugar or dolichol-pyrophosphate-oligosaccharide, for example, uridine diphospho-N-acetylglucosamine (UDP-GlcNAc), or derivatives or analogs thereof.

The acceptor or sugar donor may be labeled with a detectable substance as described herein, and the interaction of the C2 mutant polypeptide of the invention with the acceptor and sugar donor will give rise to a detectable change. The detectable change may be calorimetric, photometric, radiometric, potentiometric, etc.

An acceptor may be directly or indirectly coupled to a carrier (e.g., solid phase carrier) or support.

In certain embodiments, a Core 2 mutant polypeptide is reacted with the acceptor and sugar donor at a pH and temperature effective for the polypeptide to transfer the sugar donor to the acceptor, and where the products of the reaction are detected. One of the acceptor or donor may be labeled, to produce a detectable change or a product of the reaction may be converted to a secondary product that is readily measured. It is preferred to use a buffer with the acceptor and sugar donor to maintain the pH within the pH range effective for the polypeptides. The buffer, acceptor, and sugar donor may be used as an assay composition. Other compounds such as EDTA and detergents may be added to the assay composition.

A Core 2 mutant polypeptide of the invention may be used in high throughput methods for evaluating modulators of Core 2 GlcNAcT. For example, C2 mutant polypeptide may be used in the high throughput screening technique described in PCT/CA99/00550 (WO 99/64378) or it may be used in a radiometric or luminometric assay.

By way of example, a solid-phase bioassay is provided that involves coupling a carbohydrate acceptor for a Core 2 β-1,6-N-acetylglycosaminyl transferase enzyme to a polymer and coating onto a carrier or support; a C2 mutant polypeptide, a sugar nucleotide donor labeled with a detectable substance, and a test compound are added; and, the detectable change produced by the detectable substance is measured.

Therefore, in certain embodiments, the invention provides a solid-phase bioassay comprising the steps of (a) coupling a carbohydrate acceptor for a Core 2 β-1,6-N-acetylglycosaminyl transferase enzyme to a polymer and coating it onto a carrier or support; (b) adding a C2 mutant polypeptide of the invention, a sugar nucleotide donor labeled with a detectable substance, and a test compound; and, (c) detecting a detectable change produced by the detectable substance.

Examples of polymers to which an acceptor may be coupled include, but are not limited to polyacrylamide. The carrier or support may be for example nitrocellulose, or glass, gabbros, or magnetite. The support material may have any possible configuration including spherical (e.g., bead), cylindrical (e.g., inside surface of a test tube or well, or the external surface of a rod), or flat (e.g., sheet, test strip).

Examples of detectable substances include, but are not limited to, radioisotopes (e.g., ³H, ¹⁴C, ³⁵S, ¹²⁵I, ¹³¹I), fluorescent labels (e.g., FITC, rhodamine, lanthanide phosphors, quantum dots, etc.), luminescent labels such as luminol, enzymatic labels (e.g., horseradish peroxidase, beta.-galactosidase, luciferase, alkaline phosphatase, acetylcholinesterase), and biotinyl groups (which can be detected by marked avidin e.g., streptavidin containing a fluorescent marker or enzymatic activity that can be detected by optical or calorimetric methods). In an embodiment of the invention, the detectable substance is a radioactive material, most preferably tritium.

In an aspect of the invention, the assay involves coupling carbohydrate acceptors to a polymer (e.g., polyacrylamide) and coating onto a carrier, such as the surface of 96 well plastic plates. The Core 2 β-1,6-N-acetylglycosaminyl transferase reaction is performed with the C2 mutant polypeptide and a tritiated sugar-nucleotide donor, followed by washing, addition of scintillation counting fluid, and measurement of radioactivity with a β-counter.

The reagents suitable for applying the methods of the invention to evaluate compounds that modulate a Core 2 β-1,6-N-acetylglycosaminyl transferase may be packaged into convenient kits providing the necessary materials packaged into suitable containers. The kits may also include suitable supports useful in performing the methods of the invention.

The kits may be used to carry out the screening methods described herein. In one embodiment, the kits comprise a C2 mutant polypeptide of the invention and substrates for a Core 2 β-1,6-N-acetylglycosaminyl transferase. The substrates typically include an acceptor and a sugar donor for a Core 2 β-1,6-N-acetylglycosaminyl transferase.

In certain embodiments, the kits are used for the detection and identification of modulators of Core 2 β-1,6-N-acetylglycosaminyl transferase using high-throughput methods. For example, the kits can be used in the high-throughput method described in PCT PCT/CA99/00550 (WO 99/64378).

Compositions and Treatments

The substances or compounds identified by the methods described herein, and antibodies of the invention may be used for modulating the biological activity of a Core 2 β-1,6-N-acetylglycosaminyl transferase, and they may be used in the treatment of conditions mediated by Core 2 β-1,6-N-acetylglycosaminyl transferases. In particular, they may be used to modulate cellular adhesion associated with a number of disorders including inflammatory disorders and cancer.

The term “inflammatory” refers to reactions of both the specific and non-specific defense systems. A specific defense system reaction is a specific immune system reaction to an antigen. Examples of these reactions include antibody response to antigens such as viruses, and delayed-type hypersensitivity. A non-specific defense system reaction is an inflammatory response mediated by leukocytes (including macrophages, eosinophils, and neutrophils) generally incapable of immunological memory. Examples of non-specific reactions include the immediate swelling after a bee sting, and the collection of peripheral mononuclear leukocytes at sites of bacterial infection (pulmonary infiltrates in bacterial pneumonia and pus formation in abscesses).

Treatable disorders include rheumatoid arthritis, post-ischemic leukocyte-mediated tissue damage (reperfusion injury), frost-bite injury or shock, acute leukocyte-mediated lung injury (e.g., adult respiratory distress syndrome (ARDS)), asthma, traumatic shock, septic shock, nephritis, and acute and chronic inflammation including atopic dermatitis, psoriasis, neurotoxicity related to aberrent inflammation, and inflammatory bowel disease. Various platelet-mediated pathologies such as atherosclerosis and clotting can also be treated. The substances and compounds may be useful in minimizing tissue damage accompanying thrombotic disorders. For example, the substances, compounds, antibodies etc. can be of therapeutic value in individuals who have recently experienced stroke, myocardial infarctions, deep vein thrombosis, pulmonary embolism, etc. or in pre-thrombolytic therapy. Inhibitors of Core 2 transferase may be useful in reducing angiogenesis as well as leukocyte adhesion and entry into inflamed tissue.

A substance, compound, or antibody may be used to treat the secondary effects (e.g., pathological tissue destruction, and/or widespread microcirculatory thrombi and diffuse inflammation) of septic shock or disseminated intravascular coagulation (DIC). Substances, compounds, and antibodies herein may inhibit leukocyte emigration and mitigate tissue damage.

A substance, compound, or antibody may also be useful in treating traumatic shock and associated acute tissue injury. Inhibitory substances, compounds, and antibodies may be administered locally or systemically to control tissue damage associated with injuries.

The substances and compounds identified by the methods described herein, and antibodies may be useful in the prevention and treatment of tumors. Tumor metastasis may be inhibited or prevented by inhibiting the adhesion of circulating cancer cells. The substances, compounds, and antibodies of the invention may be especially useful in the treatment of various forms of neoplasia such as leukemias, lymphomas, melanomas, adenomas, sarcomas, and carcinomas of solid tissues in patients. In particular the composition may be used for treating malignant melanoma, pancreatic cancer, cervico-uterine cancer, cancer of the liver, kidney, stomach, lung, rectum, breast, bowel, gastric, thyroid, neck, cervix, salivary gland, bile duct, pelvis, mediastinum, urethra, bronchogenic, bladder, esophagus and colon, and Kaposi's Sarcoma.

The substances and compounds identified by the methods described herein, and antibodies of the invention may be used in the prevention and treatment of a cardiovascular disorder. Such a disorder may include, but is not limited to, arteriosclerosis including atherosclerosis and nonatheromatus arteriosclerosis, hypertension, stroke, coronary artery disease, ischemia, myocardial infarction, angina pectoris, cardiac arrhythmias, sinoatrial node blocks, atrioventricular node blocks, chronic hemodynamic overload, and aneurysm.

Liver disorders that may be prevented or treated using the substances or compounds identified by the methods described herein and antibodies of the invention, include chronic hepatitis, cancer of the liver, hepatic cirrhosis, cystic disease of the liver, Gilbert's Syndrome, Hepatitis A, B, or C, and toxic insults to the liver.

Other conditions that are treatable with a substance or compound identified in accordance with the methods described herein, and antibodies of the invention are proliferative disorders (e.g., microbial or parasitic infections), diabetes, skeletal muscle disorders, cardiomyopathy, and kidney disorders (e.g., polycystic kidney disease, glomerulonephritis). They may also be used to modulate T-cell activation and immunodeficiency due to the Wiskott-Aldrich syndrome or AIDS, or to stimulate hematopoietic progenitor cell growth, and/or confer protection against chemotherapy and radiation therapy in a subject.

Accordingly, the substances, antibodies, and compounds may be formulated into pharmaceutical compositions for administration to subjects in a biologically compatible form suitable for administration in vivo. By “biologically compatible form suitable for administration in vivo” is meant a form of the substance to be administered in which any toxic effects are outweighed by the therapeutic effects. The substances can be administered to living organisms including humans, and animals. Administration of a therapeutically active amount of the pharmaceutical compositions of the present invention is defined as an amount effective, at dosages and for periods of time necessary to achieve the desired result. For example, a therapeutically active amount of a substance may vary according to factors such as the disease state, age, sex, and weight of the individual, and the ability of antibody to elicit a desired response in the individual. Dosage regime can be adjusted to provide the optimum therapeutic response. For example, several divided doses may be administered daily or the dose may be proportionally reduced as indicated by the exigencies of the therapeutic situation.

The active substance may be administered in a convenient manner such as by injection (subcutaneous, intravenous, etc.), oral administration, inhalation, transdermal application, or rectal administration. Depending on the route of administration, the active substance may be coated in a material to protect the compound from the action of enzymes, acids and other natural conditions that may inactivate the compound.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the substances or compounds in association with one or more pharmaceutically acceptable vehicles or diluents, and contained in buffered solutions with a suitable pH and, in certain embodiments, iso-osmotic with the physiological fluids.

After pharmaceutical compositions have been prepared, they can be placed in an appropriate container and labeled for treatment of an indicated condition. For administration of a modulator (e.g., an inhibitor) of a Core 2 β-1,6-N-acetylglycosaminyl transferase, such labeling would include amount, frequency, and method of administration.

Therapeutic efficacy and toxicity of compounds, substances, and antibodies may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The therapeutic index is the dose ratio of therapeutic to toxic effects and it can be expressed as the ED₅/LD₅₀ ratio. Pharmaceutical compositions which exhibit large therapeutic indices are preferred.

Methods for Preparing Oligosaccharides

The invention also provides a method for preparing an oligosaccharide comprising contacting a reaction mixture comprising an activated GlcNAc and an acceptor in the presence of a C2 mutant polypeptide of the invention.

Examples of acceptors for use in the method for preparing an oligosaccharide ar saccharides, oligosaccharides, polysaccharides, glycopeptides, glycopolypeptides, or glycolipids which are either synthetic with linkers at the reducing end or naturally occurring structures, for example, an asialo-agalacto-fetuin glycopeptide or an asialo mucin. The activated GlcNAc may be part of a nucleotide-sugar, a dolichol-phosphate-sugar, or dolichol-pyrophosphate-oligosaccharide.

In an embodiment of the invention, the oligosaccharides are prepared on a carrier that is non-toxic to a mammal, in particular a human such as a lipid isoprenoid or polyisoprenoid alcohol. An example of a suitable carrier is dolichol phosphate. The oligosaccharide may be attached to a carrier via a labile bond allowing for chemical removal of the oligosaccharide from the lipid carrier. In the alternative, the C2 mutant polypeptide may be used to transfer the oligosaccharide from a lipid carrier to a polypeptide.

### EXAMPLES

The following examples are offered to illustrate, but not to limit the claimed invention.

### Example 1

### Preparation of C2 Mutants by Site-Directed Mutagenesis

Double stranded plasmid DNA from an S-tagged human wild type Core 2 GlcNAcT1 construct (SEQ ID NO: 29) and from a FLAG-tagged mouse wild type Core 2 GlcNAcT1 construct (SEQ ID NO: 30) was isolated using the Gibco BRL® plasmid midi plasmid prep system. The human or mouse Core2 cysteine at position 217 was mutated to either a serine or alanine using the QuickChange™ site directed mutagenesis kit from Stratagene® following manufacturer's instructions. Each mutated construct was sent for sequencing to confirm each mutation as well as the rest of the wild type sequence. Plasmid preparation for each mutated construct was performed and then used for transient transfections into COS-7, CHO, or CHO—S cell lines using Lipofectamine2000™ according to manufacturer's instructions.

Details of the preparation of the human and mouse Core 2 mutants are described below.

Human Core 2 GlcNAcT1 Mutant

The human Core 2 GlcNAcT1 cDNA (subcloned into a modified pTriEX-4 vector) was mutated at the nucleotide level such that a G was replaced with a C nucleotide to generate a protein where a Cysteine was mutated to a Serine amino acid at position 217. These experiments were performed following the instructions of the QuickChange site-directed mutagenesis kit from Stratgene. The vector (HC2Lcys217Ser) was transiently transfected into COS-7 using Lipofectamine2000™ (Invitrogen) following the manufacturer's instructions. COS-7 cells were split 1/8 to 1/10 dilution into T75 cm² flasks the day before transfection. The cells were incubated in D-MEM+10% FBS for at least 16 hours at 37° C., in 5% CO₂ to ensure the cells were 80-90% confluent and that there was efficient cell attachment on the day of transfection. Prior to transfection, the media was replaced with 20 ml D-MEM, 10% FBS, 0.1 mM non-essential amino acids and 2 mM L-Glutamine. Two solutions were prepared for transfection whereby the first solution (solution A) was composed of 32.25 μg of either pTRI-EX™ (Novagen) N-terminal S-tagged wild-type or mutant (Cys²¹⁷Ser) human Core 2 GlcNAcT1 cDNA being added to 2.5 ml of OPTI-media™ (GIBCO, BRL). The second solution (Solution B) contains 62.46 μl of Lipofectamine2000™ (Invitrogen) mixed with 2.5 ml OPTI-media™ (GIBCO/BRL). Solution B was allowed to sit at room temperature for 5 minutes before mixing the two solutions together. The mixed Solutions A and B were incubated at room temperature for 20 minutes before adding them to the COS-7 cells. The COS-7 cells/T75 cm² flask was then incubated at 37° C. in 5% CO₂ for at least 48 hours.

Forty-eight hours post transfection, media were collected and the COS-7 transfected cells were harvested with a cell scrapper to determine Core 2 GlcNAcT1 activity. This was followed by Western blot analysis. The scrapped cells were transferred to Eppendorf tubes and spun at low speed (600×g) between 3 washing steps with 1×PBS. Subsequently, the cells were lysed in 0.1 M Tris-HCl (pH 8.0), 0.1% Triton X-100, and 5 μl assayed for Core 2 GlcNAcT1 activity using a standard Core2 assay described herein. Similarly, the media was also spun at low speed (600×g) to remove dead cell debris.

One milliliter of transfected media was mixed with 2 mls of 1× Bind/Wash (20 mM Tris-HCl pH7.5, 0.15MNaCl, 0.1% Triton X-100) buffer™ (Novagen) and 1 ml of S-protein (50% slurry in 50 mM Tris-HCl, pH7.5, 150 mM NaCl, 1 mM EDTA, 0.02% sodium azide) Agarose™ (Novagen). The mixture was incubated at room temperature with gentle shaking to ensure efficient binding between the N-terminal S-tagged wild-type or mutant (Cys 27Ser) human Core 2 GlcNAcT1 and the S-protein Agarose. The mixture was spun at low speed (500×g) between 3 washing steps with 10 mls of 1×1×Bind/Wash buffer ™ (Novagen). The S-protein Agarose was resuspended in 500 μl of 1× Bind/Wash buffer™ (Novagen) and 25 μl was subjected for Western-blot analysis.

Approximately 200 μl of S-protein agarose/Core 2 GlcNAcT1 suspension was separated into a clean eppendorf tube and the thrombin reaction was set up utilizing the Biotinylated Thrombin™ (Novagen). The optimal conditions were previously determined to be 2.5 units/3 μl (1× stock concentration, 0.83 units/ul) of Biotinylated Thrombin™ (Novagen) incubated on a shaker at room temperature for up to 2 hours. Following a 2 hour incubation, the reaction mixture was spun at low speed (500×g) for 5 minutes and the supernatent (eluate) was transferred to a clean tube. The pellet was washed 2 times with 400 μl of 1× Bind/Wash buffer™ (Novagen) between spinning at low speed (500×g) and all the washes were added into the eluate. The removal of Biotinylated Thrombin™ (Novagen) was achieved by adding 200 μl of Streptavidin (50% slurry in phosphate buffer, pH7.5 0.02% sodium azide) Agarose™ (Novagen) into the pooled supernatant and incubated at room temperature with shaking for 10 minutes. The Streptavidin Agarose™ (Novagen) was removed by spinning the suspension at low speed (500×g) and the supernatant was then transferred to a 2 ml eppendorf tube. The pellet/Streptavidin Agarose™ (Novagen) was washed 2 times with 200 μl of 1× Bind/Wash buffer™ (Novagen) between spinning at low speed (500×g) and again all supernatants were pooled together in the same 2 ml eppendorf tube. Characterization analysis of both the wild-type and mutant (Cys 27Ser) human Core 2 GlcNAcT1 enzyme was performed.

Mouse Core 2 GlcNAcT1 Mutant

The mouse Core 2 GlcNAcT1 coding sequence, nucleotides 100-1287, was subcloned into a pFLAG-CMV-3 expression vector (Sigma, St. Louis, Mo., USA) in a Bgl II-XbaI insert site.

To generate a nucleic acid encoding the mouse C217S mutant, the mouse Core 2 GlcNAcT1 coding sequence, nucleotides 100-1287, subcloned into a pFLAG-CMV-3 expression vector, was mutated at base pair 650. A G nucleotide was exchanged for a C nucleotide to encode a serine residue with the QuickChange site-directed mutagenesis kit from Stratagene (La Jolla, Calif.). The mutagenic primer had the following sequence: 5′-TAC TTG ATC AAT CTC TCT GGT ATG GAT TTC CCT (SEQ ID NO:26). The underlined sequence encodes the Cys-Ser exchange.

To generate a nucleic acid encoding the mouse C217T mutant, the mouse Core 2 GlcNAcT1 coding sequence, nucleotides 100-1287, subcloned into a pFLAG-CMV-3 expression vector, was mutated at base pairs 649 and 650. The T and G nucleotides were exchanged for A and C nucleotides with the QuickChange site-directed mutagenesis kit from Stratagene. The mutagenic primer had the following sequence: 5′-TAC TTG ATC AAT CTC ACT GGT ATG GAT TTC CCT (SEQ ID NO:27). The underlined sequence encodes the Cys-Thr exchange.

To generate a nucleic acid encoding the mouse C217A mutant, the mouse Core 2 GlcNAcT1 coding sequence, nucleotides 100-1287, subcloned into a pFLAG-CMV-3 expression vector, was mutated at base pairs 649 and 650. The T and G nucleotides were exchanged for G and C nucleotides to encode an alanine residue with the QuickChange site-directed mutagenesis kit from Stratagene. The mutagenic primer had the following sequence: 5′-TAC TTG ATC AAT CTC GCT GGT ATG GAT TTC CCT (SEQ ID NO:28). The underlined sequence encodes the Cys-Ala exchange.

Each mutant construct was transfected and expressed in CHO—S or COS-7 cells. Cells were incubated with DMEM culture media (Invitrogen, Burlington, Calif.) containing 10% FBS and 0.1 mM non-essential amino acid (Invitrogen, Burlington, Calif.) to ensure the cells were confluent. The culture medium was changed one hour before transfection to activate the cells. Identical volumes of two solutions were prepared for transfection. The first solution (solution A) was 730 ng/ml of either pFLAG-CMV-3™ (Sigma) N-terminal FLAG-tagged wild-type or mutant (C217S) mouse Core 2 GlcNAcT1 cDNA in OPTI-MEM I™ (GIBCO, BRL). The second solution (Solution B) was 1.37 μl/ml of Lipofectamine2000™ (Invitrogen) in OPTI-MEM I™ (GIBCO, BRL). Solution B was incubated at room temperature for 5 minutes before mixing with Solution A. The mixed Solutions A and B were incubated at room temperature for 20 minutes before adding them to COS-7 or CHO—S cells. The COS-7 cells or CHO—S cells were then incubated at 37° C. in 5% CO₂ for at least 20 hours. Although the amount of FLAG-tagged wild-type or mutant (C217S) mouse Core 2 GlcNAcT1 cDNA and Lipofectamine2000™ depended on the scale of the transfection, a ratio of 0.61 μl Lipofectamine2000™ to 320 ng-640 ng FLAG-tagged wild-type or mutant (C217S) mouse Core 2 GlcNAcT1 cDNA was maintained.

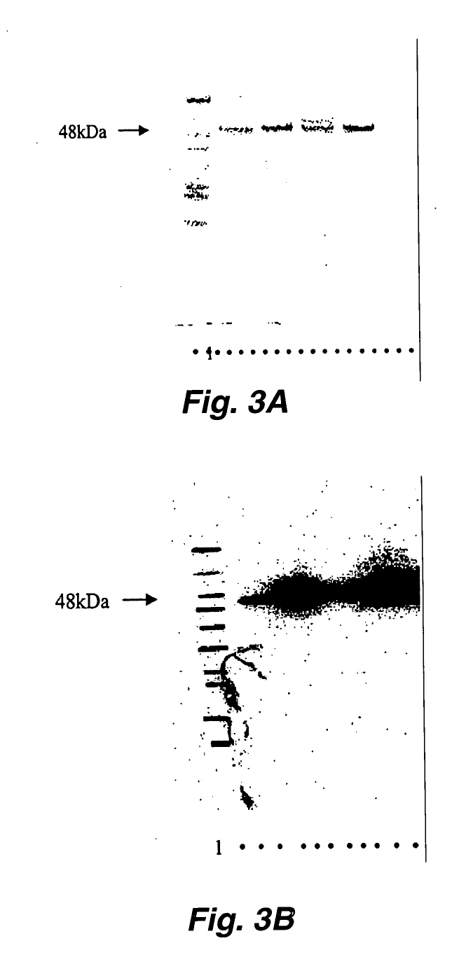

The cells were washed three times with D-PBS, and the culture media was then exchanged with CHO—SFM-II (Invitrogen, Burlington, Calif.). The cells were incubated for 24 hours to collect the serum free culture supernatant that contained Core 2 GlcNAcT1 wild type or C217S mutant polypeptide. The CHO—SFM-II supernatants were collected every 24 hours and fresh CHO—SFM-II medium was added until the cells lost their productivity of Core 2 GlcNAcT1 wild type or C217S mutant polypeptide. Core 2 GlcNAcT1 activity was identified in the cell culture supernatants, and the presence of Core 2 GlcNAcT1 polypeptides was verified by SDS-PAGE and Western blotting with peroxidase conjugated anti-FLAG M2 antibody (Sigma) as shown in FIGS. 3A and 3B.

To purify the FLAG-tagged C217S mouse Core 2 GlcNAcT1 mutant polypeptide, 1 L to 5 L of serum free culture supernatant was collected from large scale transfections of CHO—S cells or COS-7 cells. The collected culture supernatant was applied to 25 ml of anti-FLAG M1 affinity beads (Sigma). The beads and supernatants were incubated for one hour at room temperature with gentle shaking. The beads and supernatants were then transferred to a column, and the beads were washed with 5 mM CaCl₂, 150 mM NaCl and 50 mM Tris-HCl (pH7.4). With the column flow stopped, 35 ml of elution buffer (2 mM EDTA and 0.05% Tween 20 in 50 mM Tris-HCl, pH 7.4) was added to the beads and incubated for thirty minutes with gentle mixing to detach the mutant polypeptide from the beads. Eluate comprising FLAG-tagged mutant polypeptide was collected by opening the column flow. The elution procedure was repeated three times. A total of 105 ml eluate was collected and then concentrated. The solvent was replaced with 150 mM NaCl and 50 mM Tris-HCl(pH7.4) with Centricon-Plus80 (Millipore, Billerica, Mass., USA).

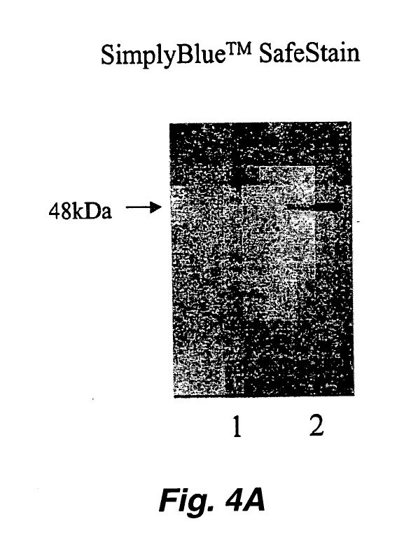

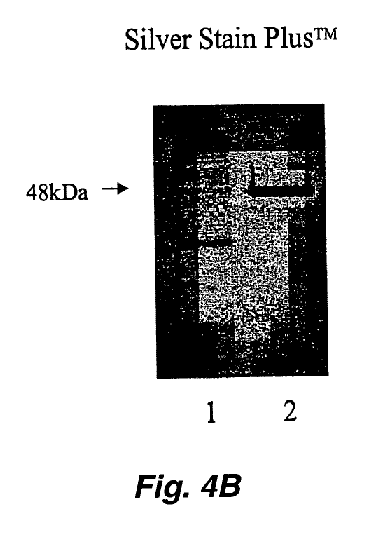

The purity of FLAG-tagged C217S mouse Core 2 GlcNAcT1 polypeptide was estimated to be greater than 99% by both SimplyBlue™ SafeStain (Invitrogen) and Silver Stain Plus (Bio-Rad) as shown in FIGS. 4A and 4B. The FLAG-tagged C217S mouse Core 2 GlcNAcT1 mutant polypeptide migrated as a single band at 48 kDa. Protein quantification of the FLAG-tagged C217S mouse Core 2 GlcNAcT1 mutant polypeptide was performed with a BCA protein assay kit (Pierce).

Core 2 GlcNAcT1 activity was identified by the enzyme assays of Examples 2 and 3. In particular, the reaction was carried out in a 40 μl solution containing 30 mM MES buffer (pH 6.7), 1.0 mM UDP-GlcNAc, 1.0 μCi UDP-6-[³H]GlcNAc, 0.5 mM Galβ1-3GalNAcα-pNp, 0.25 mg/ml BSA and 0.5 ng purified FLAG-tagged C217S mouse Core 2 GlcNAcT1 polypeptide. The reaction was incubated for 30 minutes at 37° C. and stopped by the addition of 500 μl of water. The reaction product Galβ1-3([³H]GlcNAcβ1-6)GalNAcα-pNp was separated from unreacted UDP-6-[³H]GlcNAc by solid phase extraction using Waters Sep-Pak columns. Samples were eluted from the column with ethanol, added to 10 ml of scintillation fluid and radioactivity (DPM) was measured. Specific activity was calculated as the amount of reaction product per one-minute reaction and per one mg polypeptide.

Purified FLAG-tagged C217S mouse Core 2 GlcNAcT1 polypeptide was obtained with a specific activity of up to 15.7 micromole/min/mg. For comparison, the specific activity of FLAG-tagged mouse wild type Core 2 GlcNAcT1 polypeptide in the presence of 1 mM 1,4-dithiolthreitol (DTT) was similar, 9.2 micromole/min/mg. Purification of FLAG-tagged wild type mouse Core 2 GlcNAcT1 polypeptide followed the method described above for the FLAG-tagged mouse C217S Core 2 GlcNAcT1 mutant polypeptide using 40 ml cell culture supernatant. The purity of the FLAG-tagged mouse wild type Core 2 GlcNAcT1 polypeptide is provided in FIGS. 3A and 3B.

### Example 2

### Human and Mouse Wild-Type and Mutant Core 2 GlcNAcT1 Properties

A. Human and Mouse Core 2 GlcNAcT1 Wild-Type Properties.

The activity of wild-type human and mouse Core 2 GlcNAcT1 was observed to increase after the addition of 1,4-dithiothreitol (DTT) or other reducing agents following the storage of expressed and purified recombinant enzyme. Iodoacetamide, 5,5′-dithiobis(2-nitrobenzoic acid) (DTNB), and N-ethylmaleimide causes inhibition of wild-type human and mouse Core 2 GlcNAcT1s. The heavy metals Hg²⁺ and Zn²⁺ also inhibit wild-type human and mouse Core 2 GlcNAcT1s. These results indicate that wild-type human and mouse Core 2 GlcNAcT1s have a reduced cysteine amino acid residue that is necessary for proper enzyme function. The blocking of this residue by oxidation, alkylation, or heavy metal binding causes inhibition of the enzyme.

B. Effects of Sulfhydryl Reagents on Mouse Core 2 GlcNAcT1

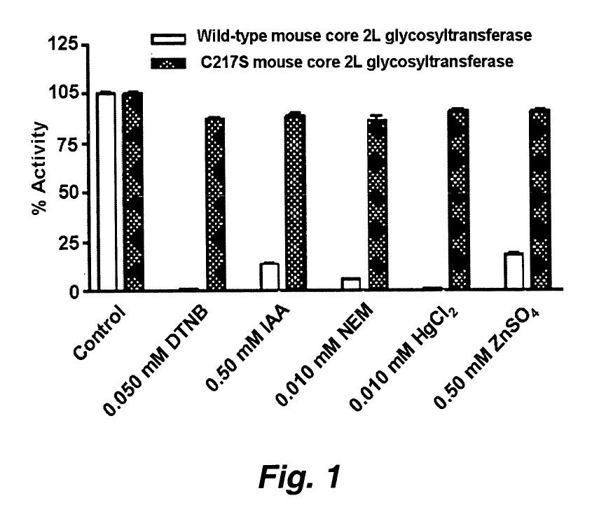

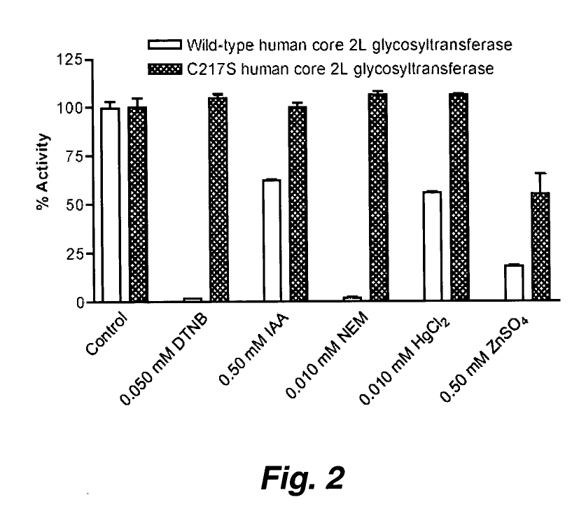

Wild-type and C217S, human and mouse, Core 2 GlcNAcT1s were dissolved in an aqueous solution containing 30 mM 2-morpholinoethanesulfonic acid (MES) buffer (pH 6.7) and 1.0 mg/ml bovine serum albumin (BSA). Iodoacetamide (IAA), 5,5′-dithiobis(2-nitrobenzoic acid) (DTNB), N-ethylmaleimide (NEM), HgCl₂ and ZnSO₄ were added at the concentrations listed on FIGS. 1 and 2. 1.0 mM UDP-GlcNAc, 0.50 mM Gal(β1-3)GalNAcα-pnp, (3H)UDP-GlcNAc (0.50 μCi) and 30 mM MES buffer (pH 6.7) were added as well. The solutions were incubated at 37° C. for 2.0 hours. The (³H)UDP-GlcNAc and (³H)GlcNAc(β1-6)Gal(β1-3)GalNAcα-pnp were separated by solid-phase extraction and the DPM of (³H)Gal(β1-3) GlcNAc(β1-6)GalNAcα-pnp were counted by liquid scintillation. A solution containing all components with exception of enzymes was prepared to measure background levels. The % activity was calculated as follows: (DPM_{inhibitor}−DPM_{background})/(DPM_{control}−DPM_{background})*100. All assays were performed in duplicate. The results are plotted in FIGS. 1 and 2.

The activity of C217S human and mouse Core 2 GlcNAcT1 were not inhibited by relatively high concentrations of the thiol-reactive reagents iodoacetamide, 5,5′-dithiobis(2-nitrobenzoic acid) (DTNB), and N-ethylmaleimide (NEM). Similarly, relatively high concentrations of Hg²⁺ and Zn²⁺ did not affect the mouse enzyme while the human enzyme was insensitive to Hg²⁺ only. These results indicate that thiol oxidation; alkylation or some heavy metal binding no longer inhibits C217S human and mouse Core 2 GlcNAcT1s.

Data showing the effects of anti-thiol reagents on wild-type and C217S, human and mouse, Core 2 GlcNAcT1s is shown in FIG. 1 and FIG. 2.

C. Human and Mouse Core 2 GlcNAcT1 C217S Properties.

Methods:

Standard Enzyme Assay.

Standard enzyme assays for wild-type and mutant (217Cys→Ser) Core 2 GlcNAcT1 were carried out in a 30 μl solution containing 30 mM 2-morpholinoethanesulfonic acid (MES) buffer (pH 6.7), 1.0 mM UDP-GlcNAc (donor substrate), 1.0 μCi UDP-6-[3H]GlcNAc, 0.50 mM (Galβ1-3)GalNAcα-pNp (acceptor substrate or AS), 1 mg/ml BSA, and 15-25 μU (1 unit=1 micromole/min) per assay of recombinant Core 2 GlcNAcT1 protein. The reaction was incubated for 2.0 hours at 37° C. and stopped by the addition of 460 μl of water. Reactions were frozen at −20° C. if they were not processed immediately. The reaction product Galβ1,3 [³H]GlcNAc(β1,6)GalNAcα-pNp was separated from unreacted UDP-6[³H]GlcNAc by solid phase extraction using Waters Sep-Pak columns. Samples were eluted from the column with ethanol, added to 10 ml of scintillation fluid and radioactivity (DPM) was determined.

Kinetic Characterization Assays

Km values were determined by running the standard assay conditions with the exception of the titration of the donor substrate UDP-GlcNAc concentration (for K_{m,DS}) or AS concentration (for K_{m,AS}). The IC₅₀ values for the products UDP and GlcNAc(β1-6)Gal(β1-3)GalNAcα-pNp were determined by running the standard assay conditions and adding titrated product. The mechanism was determined by titrating both donor and acceptor substrates at the same time.

Results:

Kinetic Comparison of Wild-Type and C217S Mouse Core 2 GlcNAcT1

The Kₘ value for the Core 2 GlcNAcT1 donor substrate, UDP-GlcNAc, is 1.5 mM for the wild-type enzyme and 1.5 mM for the C217S enzyme. The Kₘ value for the Core 2 GlcNAcT1 acceptor substrate, Gal(β1-3)GalNAcα-pNp, is 0.15 mM for the wild-type enzyme and 0.13 mM for the C217S enzyme. Wild-type and C217S Core 2 GlcNAcT1s maintain an Ordered Bi Bi mechanism and the K_{D} for the donor substrate is 0.11 mM for both enzymes. The inhibitory potential for the Core 2 GlcNAcT1 products, UDP and Gal(β1-3) GlcNAc(β1-6)GalNAcα-pNp, are the same for both enzymes. Wild-type and C217S Core 2 GlcNAcT1s do not use UDP-Glc as a substrate. Compounds that inhibit wild-type Core 2 GlcNAcT1 in the presence of DTT also inhibit C217S Core 2 GlcNAcT1. Compounds that do not inhibit wild-type Core 2 GlcNAcT1 in the presence of DTT do not inhibit C217S Core 2 GlcNAcT1.

Kinetic Comparison of Human Wild-Type and C217S Core 2 GlcNAcT1

The Kₘ value for the Core 2 GlcNAcT1 donor substrate, UDP-GlcNAc, is 1.2 mM for the wild-type enzyme and 1.1 mM for the C217S enzyme. The Kₘ value for the Core 2 GlcNAcT1 acceptor substrate, Gal (β1-3)GalNAcα-pnp, is 0.34 mM for the wildtype enzyme and 0.32 mM for the C217S enzyme. Wild-type and C217S Core 2 GlcNAcT1s maintain the same kinetic mechanism and the inhibitory potential for the Core 2 GlcNAcT1 products, UDP and Gal(β1-3) GlcNAc(β1-6)GalNAcα-pnp, is the same for both enzymes. Wild-type and C217S Core 2 GlcNAcT1s do not use UDP-Glc as a substrate.

### Example 3

### Screening Method

Core 2 GlcNAcT enzyme catalyzes the trans-glycosylation reaction of ³H radiolabeled GlcNAc from UDP-GlcNAc to the acceptor substrate Galβ1,3GalNAcα-pNitrophenyl. Radiolabeled product is isolated via solid-phase extraction and counted by liquid scintillation. The rate of product formation is then calculated. Test compounds are added to the Core 2 GlcNAcT reaction mixture to determine their inhibitory potential. Trans-glycosylation rates are measured and compared to an uninhibited control to obtain a percent control activity or “inhibitory rate %”. A recombinant mutant enzyme of the invention is used to assay inherent inhibitor potential.

A particular high throughput protocol is set out below.

A stock solution of the Core 2 acceptor Galβ1,3GalNAcβ-pNitrophenyl (GGCAN) was prepared by re-suspending the acceptor in water and vortexing to mix the solution. The acceptor solution was stored at −20° C. 96-well microtitre plates (MicroWell Plates, V-Bottom, Nalge Nunc). Recombinant C217S human and mouse Core 2 GlcNAcT1s were prepared as described herein.

A Core 2 transferase assay to screen for inhibitors of the enzyme involved addition of a recombinant assay mixture per reaction in wells or microtitre plates. The assay mixture consisted of 10 μl of test compound, 10 μl of a 3× assay mixture (made as a 10 ml stock solution consisting of 0.9 ml 1M MES pH 6.7, 0.3 ml 0.5M EDTA, 0.3 ml 100 mM UDP-GlcNAc, 1 mCi UDP-6-[³H]GlcNAc, 3 ml 5 mM GGNAN, and 5.5 ml water), and 10 μl of recombinant C217S human or mouse Core 2 GlcNAcT1 (15-25 μU [1 unit=1 micromole/min]) per reaction well. After incubating the plates at 37° C. for 2 hours with agitation, the reactions were stopped by adding 200 μl of water to each well. The plates were stored at −20° C. if isolation of product was not performed within 1 hour.

Product was isolated using OASIS HLB 96-well plates (Waters), according to the manufacturer's instructions. Elution was carried out with ethanol, after which plates were completely dried prior to resuspending the radioactive product with scintillation fluid. Radioactivity in eluates was counted using a MicroBeta TriLux microplate liquid scintillation counter (Wallac).

Enzyme activity (nmole product/hr) was determined using the following formula:
enzyme activity=DPM-background/2.22×10⁶×30 nMol UDP-GlcNAc/incubation time Inhibitory rate %: (enzyme activity in the presence of test compound/enzyme activity in the absence of test compound)×100

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the cell lines, vectors, methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

1. An isolated Core 2 GlcNAcT polypeptide wherein amino acid residue 217 is any amino acid other than cysteine.

2. An isolated Core 2 GlcNAcT polypeptide of claim 1 wherein amino acid residue 217 is any amino acid other than cysteine or serine.

3. An isolated Core 2 GlcNAcT polypeptide of claim 1 wherein amino acid residue 217 is serine, threonine or alanine.

4. An isolated Core 2 GlcNAcT polypeptide comprising an amino acid sequence that differs from that set forth in SEQ ID NO:3 or SEQ ID NO:7 at amino acid residue 217.

5. An isolated Core 2 GlcNAcT polypeptide of claim 3 wherein amino acid residue 217 is not serine.

6. An isolated Core 2 GlcNAcT polypeptide comprising amino acid sequence WX¹YX²INX³X⁴GX⁵DFP (SEQ ID NO:31), wherein each of X¹, X², X³ and X⁵ is independently any amino acid residue and X⁴ is any residue other than cysteine.

7. The isolated polypeptide of claim 6 wherein X⁴ is not serine.

8. The isolated polypeptide of claim 6 wherein X⁴ is selected from the group consisting of serine, threonine, or alanine.

9. An isolated polypeptide of claim 6 wherein the amino acid sequence is WKYLINLX⁴GMDFP (SEQ ID NO:32).

10. The isolated polypeptide of claim 9 wherein X⁴ is selected from the group consisting of serine, threonine, or alanine.

11. An isolated polypeptide of any of claims 1 through 10 wherein said polypeptide is a human polypeptide.

12. An isolated polypeptide of any of claims 1 through 10 wherein said polypeptide is a mouse polypeptide.

13. An isolated polypeptide comprising the amino acid sequence of SEQ ID NOs:9, 11, 17, or 19.

14. A polypeptide of any of claims 1 through 10 wherein said polypeptide has Core 2 GlcNAcT activity.

15. A Core 2 GlcNAcT polypeptide that is modified at cysteine 217 and has Core 2 GlcNAcT activity.

16. A mutant polypeptide of Core 2 GlcNAcT comprising an amino acid substitution wherein a cysteine residue at position 217 is replaced by a serine residue and wherein said mutant polypeptide has increased stability compared with the wild type Core 2 GlcNAcT.

17. The mutant polypeptide of claim 16 wherein the increased stability is characterized by insensitivity to inactivation caused by oxidation, alkylation, or heavy metal binding.

18. A mutant polypeptide of Core 2 GlcNAcT comprising an amino acid substitution wherein a cysteine residue at position 217 is replaced by an alanine residue and wherein said mutant polypeptide has increased stability compared with the wild type Core 2 GlcNAcT.

19. The mutant polypeptide of claim 18 wherein the increased stability is characterized by insensitivity to inactivation caused by oxidation, alkylation, or heavy metal binding.

20. A fusion polypeptide comprising the polypeptide of any of claims 1-10, 13, 15, 17, 18, or 19 operably linked to a fusion molecule.

21. A fusion polypeptide of claim 20 wherein the fusion molecule is selected from the group consising of immunoglobulin, glutathione-S-transferase, protein A, HI, hemagglutinin, S-tag, FLAG, β-galactosidase, maltose E binding protein, GAL, HSP, LacZ, His-tag, avidin and truncated myc.

22. A fusion polypeptide comprising the amino acid sequence of SEQ ID NOs:10, 12, 18, or 20.

23. An antibody that specifically binds a polypeptide of any of claims 1-10, 13, 15, 17, 18, or 19.

24. An isolated nucleic acid molecule encoding a polypeptide of any of claims 1-10, 13, 15, 17, 18, or 19.

25. An isolated nucleic acid molecule encoding a fusion polypeptide selected from the group consisting of a fusion polypeptide comprising the polypeptide of any of claims 1-10, 13, 15, 17, 18, or 19 operably linked to a fusion molecule, and a fusion polypeptide comprising the amino acid sequence of SEQ ID NOs:10, 12, 18, or 20.

26. An isolated nucleic acid molecule comprising SEQ ID NOs:13, 15, 21, or 23.

27. An isolated nucleic acid molecule consisting essentially of SEQ ID NOs:13, 15, 21, or 23.

28. A recombinant vector comprising an isolated nucleic acid molecule selected from the group consisting of a nucleic acid encoding a polypeptide of any of claims 1-10, 13, 15, 17, 18, or 19, a nucleic acid encoding a fusion protein comprising a polypeptide of any of claims 1-10, 13, 15, 17, 18, or 19, a nucleic acid encoding a fusion polypeptide comprising the amino acid sequence of SEQ ID NOs:10, 12, 18, or 20, and a nucleic acid molecule comprising SEQ ID NOs:13, 15, 21, or 23.

29. A method of producing a recombinant vector comprising inserting into a vector a nucleic acid molecule selected from the group consisting of a nucleic acid encoding a polypeptide of any of claims 1-10, 13, 15, 17, 18, or 19, a nucleic acid encoding a fusion protein comprising a polypeptide of any of claims 1-10, 13, 15, 17, 18, or 19, a nucleic acid encoding a fusion polypeptide comprising the amino acid sequence of SEQ ID NOs:10, 12, 18, or 20, and an isolated nucleic acid molecule comprising SEQ ID NOs:13, 15, 21, or 23.

30. A host cell comprising a nucleic acid molecule selected from the group consisting of a nucleic acid encoding a polypeptide of any of claims 1-10, 13, 15, 17, 18, or 19, a nucleic acid encoding a fusion protein comprising a polypeptide of any of claims 1-10, 13, 15, 17, 18, or 19, a nucleic acid encoding a fusion polypeptide comprising the amino acid sequence of SEQ ID NOs:10, 12, 18, or 20, and an isolated nucleic acid molecule comprising SEQ ID NOs:13, 15, 21, or 23.

31. A host cell as claimed in claim 30 wherein the nucleic acid molecule is operably associated with a heterologous regulatory sequence that controls gene expression.

32. A host cell comprising a recombinant vector comprising an isolated nucleic acid molecule selected from the group consisting of a nucleic acid encoding a polypeptide of any of claims 1-10, 13, 15, 17, 18, or 19, a nucleic acid encoding a fusion protein comprising a polypeptide of any of claims 1-10, 13, 15, 17, 18, or 19, a nucleic acid encoding a fusion polypeptide comprising the amino acid sequence of SEQ ID NOs:10, 12, 18, or 20, and an isolated nucleic acid molecule comprising SEQ ID NOs:13, 15, 21, or 23.

33. A method of producing a host cell comprising transducing, transforming, or transfecting a host cell with a recombinant vector of claim 28.

34. A method for producing a polypeptide comprising:
culturing a host cell of claim 28 under conditions suitable to produce a polypeptide encoded by the nucleic acid molecule; and
recovering the polypeptide from the cell culture.

35. A method for identifying a substance that modulates the expression or biological activity of a Core 2 β1-6-N-acetylglycosaminyl transferase comprising assaying for substances that modulate the activity of a polypeptide of any of claims 1-10, 13, 15, 17, 18, or 19.

36. A method for evaluating a substance for its ability to modulate the activity of a Core 2 GlcNAcT comprising:
(a) contacting an acceptor and a sugar donor for a Core 2 GlcNAcT with a polypeptide of any of claims 1-10, 13, 15, 17, 18, or 19, in the presence of a test substance;
(b) measuring the amount of sugar donor transferred to acceptor, and
(c) carrying out steps (a) and (b) in the absence of the test substance to determine if the substance interferes with or enhances transfer of the sugar donor to the acceptor by the polypeptide.

37. A method for evaluating a test substance for its ability to modulate the activity of a Core 2 GlcNAcT comprising:
(a) contacting a Core 2 GlcNAcT acceptor and sugar donor with a polypeptide of any preceding claim in the presence of the test substance; and
(b) comparing the amount of sugar donor transferred to acceptor in step (a) to the amount of sugar donor transferred to acceptor in the absence of the test substance.

38. A method as claimed in claim 35, 36, or 37 which is a high throughput method.

39. A method as claimed in claim 36, or 37 wherein the acceptor is coupled to a solid phase carrier or support.

40. A method as claimed in claim 36, or 37 wherein the acceptor is coupled to a polymer and the polymer is coated onto a carrier or support.

41. A method as claimed in claim 36, or 37 wherein the sugar donor is labeled with a detectable substance.

42. A method of claim 41 wherein the amount of sugar donor transferred to acceptor is measured by detecting the detectable substance coupled to the solid phase carrier or support.

43. A method for preparing an oligosaccharide comprising contacting a polypeptide of any preceding claim with a Core 2 GlcNAcT acceptor and sugar donor under conditions suitable for linking the acceptor and the sugar donor.

44. The method of claim 43 wherein the sugar donor is activated GlcNAc.

45. A kit comprising a polypeptide of any of claims 1, 3, 4, 6, 8, 9, 10, 13, 15, 16, 17, 18, 19 and substrates for Core 2 β-1,6-N-acetylglycosarninyl transferase.


SEQUENCE LISTING (30 sequences)

SEQ ID NO: 1 - DNA, 2110 bp, Homo sapiens
gtgaagtgctcagaatggggcaggatgtcacctggaatcagcactaagtgattcagactttccttacttttaaatgtgctgctcttcatttcaagatgccgttgcagctctgataaatgcaaactgacaaccttcaaggccacgacggagggaaaatcattggtgcttggagcatagaagactgcccttcacaaaggaaatccctgattattgtttgaaatgctgaggacgttgctgcgaaggagacttttttcttatcccaccaaatactactttatggttcttgttttatccctaatcaccttctccgttttaaggattcatcaaaagcctgaatttgtaagtgtcagacacttggagcttgctggggagaatcctagtagtgatattaattgcaccaaagttttacagggtgatgtaaatgaaatccaaaaggtaaagcttgagatcctaacagtgaaatttaaaaagcgccctcggtggacacctgacgactatataaacatgaccagtgactgttcttctttcatcaagagacgcaaatatattgtagaaccccttagtaaagaagaggcggagtttccaatagcatattctatagtggttcatcacaagattgaaatgcttgacaggctgctgagggccatctatatgcctcagaatttctattgcgttcatgtggacacaaaatccgaggattcctatttagctgcagtgatgggcatcgcttcctgttttagtaatgtctttgtggccagccgattggagagtgtggtttatgcatcgtggagccgggttcaggctgacctcaactgcatgaaggatctctatgcaatgagtgcaaactggaagtacttgataaatctttgtggtatggattttcccattaaaaccaacctagaaattgtcaggaagctcaagttgttaatgggagaaaacaacctggaaacggagaggatgccatcccataaagaagaaaggtggaagaagcggtatgaggtcgttaatggaaagctgacaaacacagggactgtcaaaatgcttcctccactcgaaacacctctcttttctggcagtgcctacttcgtggtcagtagggagtatgtggggtatgtactacagaatgaaaaaatccaaaagttgatggagtgggcacaagacacatacagccctgatgagtatctctgggccaccatccaaaggattcctgaagtcccgggctcactccctgccagccataagtatgatctatctgacatgcaagcagttgccaggtttgtcaagtggcagtactttgagggtgatgtttccaagggtgctccctacccgccctgcgatggagtccatgtgcgctcagtgtgcattttcggagctggtgacttgaactggatgctgcgcaaacaccacttgtttgccaataagtttgacgtggatgttgacctctttgccatccagtgtttggatgagcatttgagacacaaagctttggagacattaaaacactgaccattacgggcaattttatgaacaagaagaaggatacacaaaacgtaccttatctgtttccccttccttgtcagcgtcgggaagatggtatgaagtcctctttggggcagggactctagtagatcttcttgtcagagaagctgcatggtttctgcagagcacagttagctagaaaggtgatagcattaaatgttcatctagagttaatagtgggaggagtaaaggtagccttgaggccagagcaggtagcaaggcattgtggaaagaggggaccagggtggctggggaagaggccgatgcataaagtcagcctgttccaagtgctcagggacttagcaaaatgagaagatgtgacctgtgccaaaactattttgagaattttaaatgtgaccatttttctggtatgccaataaaddcttacagcaacaaataatcaaagatacaattaatctgatattatatttgttgaaatagaaatttgattgtactataaatgatttttgtaaataatttatattctgctctaatactgtactgtgtagtgtgtctccgtatgtcatctcagggagcttaaaatgggcttgatttaacattgaaaaaaaat

SEQ ID NO: 2 - DNA, 1176 bp, Artificial
  Nucleic acid encoding recombinant fusion protein
aagcctgaatttgtaagtgtcagacacttggagcttgctggggagaatcctagtagtgatattaattgcaccaaagttttacagggtgatgtaaatgaaatccaaaaggtaaagcttgagatcctaacagtgaaatttaaaaagcgccctcggtggacacctgacgactatataaacatgaccagtgactgttcttctttcatcaagagacgcaaatatattgtagaaccccttagtaaagaagaggcggagtttccaatagcatattctatagtggttcatcacaagattgaaatgcttgacaggctgctgagggccatctatatgcctcagaatttctattgcgttcatgtggacacaaaatccgaggattcctatttagctgcagtgatgggcatcgcttcctgttttagtaatgtctttgtggccagccgattggagagtgtggtttatgcatcgtggagccgggttcaggctgacctcaactgcatgaaggatctctatgcaatgagtgcaaactggaagtacttgataaatctttgtggtatggattttcccattaaaaccaacctagaaattgtcaggaagctcaagttgttaatgggagaaaacaacctggaaacggagaggatgccatcccataaagaagaaaggtggaagaagcggtatgaggtcgttaatggaaagctgacaaacacagggactgtcaaaatgcttcctccactcgaaacacctctcttttctggcagtgcctacttcgtggtcagtagggagtatgtggggtatgtactacagaatgaaaaaatccaaaagttgatggagtgggcacaagacacatacagccctgatgagtatctctgggccaccatccaaaggattcctgaagtcccgggctcactccctgccagccataagtatgatctatctgacatgcaagcagttgccaggtttgtcaagtggcagtactttgagggtgatgtttccaagggtgctccctacccgccctgcgatggagtccatgtgcgctcagtgtgcattttcggagctggtgacttgaactggatgctgcgcaaacaccacttgtttgccaataagtttgacgtggatgttgacctctttgccatccagtgtttggatgagcatttgagacacaaagctttggagacattaaaacac

SEQ ID NO: 3 - protein, 428 aa, Homo sapiens [sequence omitted]

SEQ ID NO: 4 - protein, 392 aa, Artificial
  Recombinant fusion protein
KPEFVSVRHLELAGENPSSDINCTKVLQGDVNEIQKVKLEILTVKFKKRPRWTPDDYINMTSDCSSFIKRRKYIVEPLSKEEAEFPIAYSIVVHHKIEMLDRLLRAIYMPQNFYCVHVDTKSEDSYLAAVMGIASCFSNVFVASRLESVVYASWSRVQADLNCMKDLYAMSANWKYLINLCGMDFPIKTNLEIVRKLKLLMGENNLETERMPSHKEERWKKRYEVVNGKLTNTGTVKMLPPLETPLFSGSAYFVVSREYVGYVLQNEKIQKLMEWAQDTYSPDEYLWATIQRIPEVPGSLPASHKYDLSDMQAVARFVKWQYFEGDVSKGAPYPPCDGVHVRSVCIFGAGDLNWMLRKHHLFANKFDVDVDLFAIQCLDEHLRHKALETLKH

SEQ ID NO: 5 - DNA, 2033 bp, Mus musculus
cctggggactgcttcatctttcctgcaaactgtgtgtgcagcaaaggcagcttcactctctgctggtgttcatggctctgcctttcagggattgtgccgagctccaggtggttgtagctgcctaacataggtgctaggaaccaaaccctggtcttctagatgaagaagaagcactgtaaattcctgaacctccccacaccccggcatggtatatttgttataactaatataactccacccccacccttttcttcaacactgaggattaaacccgcgcctcatggatacaggacaagtgctcaactggctgttggaaagcctgctgggacatgtcatcgcgggatgcctggtgcttgataggaacttggcagcacaatattccctctgagcaagtacaggatgtcaccaggagtcagagcctcaacagattcaggctgcctgtgattctaaatgtgatatcttgtcccgctgataaactggcatctcacctggccacatcaggacactggaggaaacatcattggcattcaaggcctagaagattgtcccaccacaccaggaagcctctgactgttctttgaaatgctgagaaacttgtttcggaggagacttttctcttgtcctacaaaatactactttatgctccttgtcctctctttaattaccttctctgttttaagaattcatcagaagcctgaattttttagtgtcagacacttggagctggctggagatgatccttacagcaatgttaattgcaccaagattttacagggtgacccagaagaaatccagaaggtgaagcttgagatactaacagtgcaattcaagaagcgcccgaggtggacaccccatgactacataaacatgacccgtgactgtgcctctttcatcaggacacgcaaatatattgtggagccccttactaaagaagaggtaggctttccaattgcatattccattgtggttcatcataagattgaaatgcttgacaggctcctaagggccatctatatgcctcagaatttctactgcattcacgtggacagaaaagcagaggaatcctttttagccgcggtgcagggcattgcatcctgctttgataatgtctttgtggccagccagttggagagtgttgtttatgcgtcctggactcgggttaaagcagacctcaactgcatgaaggacctgtacagaatgaatgcaaactggaagtacttgatcaatctctgtggtatggatttccctattaaaaccaacctggaaattgtcaggaagctcaagtgctccacaggggaaaacaacctggaaactgagaagatgcctcccaacaaggaagaaagatggaaaaaaagatacgccgttgtcgatgggaagctgaccaacactgggatagtcaaagcaccgcccccactcaaaactcctctcttttcaggcagtgcctacttcgtggtcactagggaatatgtaggctacgtgctggaaaatgaaaatattcaaaagttgatggaatgggcacaggacacatacagcccagatgagttcctctgggccaccatccaaaggatcccagaagtccctggttctttcccctcaagcaacaagtatgacttgtcagacatgaatgccattgctaggtttgtcaagtggcagtacttcgaaggcgatgtttccaatggtgccccttatccaccgtgcagtggagtccacgtgcgctctgtgtgcgtcttcggagctggtgacttgagctggatgctgcgccaacaccacctttttgccaataagtttgacatggatgtcgacccctttgccatccagtgtttggatgagcatctgaggcgtaaagccctggagaacttagaacactaagcgctgctggcagccctggggaagataaggatgctcaaactgtactctcatctgcttcccatctctcctttggggtcctccttggggtgatgactttaaagtcttcctgtcagggaaactgcatgaatcctacagaatatagttacaggagaggctgatacctt

SEQ ID NO: 6 - DNA, 1188 bp, Artificial
  Nucleic acid encoding recombinant fusion protein
attcatcagaagcctgaattttttagtgtcagacacttggagctggctggagatgatccttacagcaatgttaattgcaccaagattttacagggtgacccagaagaaatccagaaggtgaagcttgagatactaacagtgcaattcaagaagcgcccgaggtggacaccccatgactacataaacatgacccgtgactgtgcctctttcatcaggacacgcaaatatattgtggagccccttactaaagaagaggtaggctttccaattgcatattccattgtggttcatcataagattgaaatgcttgacaggctcctaagggccatctatatgcctcagaatttctactgcattcacgtggacagaaaagcagaggaatcctttttagccgcggtgcagggcattgcatcctgctttgataatgtctttgtggccagccagttggagagtgttgtttatgcgtcctggactcgggttaaagcagacctcaactgcatgaaggacctgtacagaatgaatgcaaactggaagtacttgatcaatctctgtggtatggatttccctattaaaaccaacctggaaattgtcaggaagctcaagtgctccacaggggaaaacaacctggaaactgagaagatgcctcccaacaaggaagaaagatggaaaaaaagatacgccgttgtcgatgggaagctgaccaacactgggatagtcaaagcaccgcccccactcaaaactcctctcttttcaggcagtgcctacttcgtggtcactagggaatatgtaggctacgtgctggaaaatgaaaatattcaaaagttgatggaatgggcacaggacacatacagcccagatgagttcctctgggccaccatccaaaggatcccagaagtccctggttctttcccctcaagcaacaagtatgacttgtcagacatgaatgccattgctaggtttgtcaagtggcagtacttcgaaggcgatgtttccaatggtgccccttatccaccgtgcagtggagtccacgtgcgctctgtgtgcgtcttcggagctggtgacttgagctggatgctgcgccaacaccacctttttgccaataagtttgacatggatgtcgacccctttgccatccagtgtttggatgagcatctgaggcgtaaagccctggagaacttagaacactaa

SEQ ID NO: 7 - protein, 428 aa, Mus musculus
MLRNLFRRRLFSCPTKYYFMLLVLSLITFSVLRIHQKPEFFSVRHLELAGDDPYSNVNCTKILQGDPEEIQKVKLEILTVQFKKRPRWTPHDYINMTRDCASFIRTRKYIVEPLTKEEVGFPIAYSIVVHHKIEMLDRLLRAIYMPQNFYCIHVDRKAEESFLAAVQGIASCFDNVFVASQLESVVYASWTRVKADLNCMKDLYRMNANWKYLINLCGMDFPIKTNLEIVRKLKCSTGENNLETEKMPPNKEERWKKRYAVVDGKLTNTGIVKAPPPLKTPLFSGSAYFVVTREYVGYVLENENIQKLMEWAQDTYSPDEFLWATIQRIPEVPGSFPSSNKYDLSDMNAIARFVKWQYFEGDVSNGAPYPPCSGVHVRSVCVFGAGDLSWMLRQHHLFANKFDMDVDPFAIQCLDEHLRRKALENLEH

SEQ ID NO: 8 - protein, 395 aa, Artificial
  Recombinant fusion protein
IHQKPEFFSVRHLELAGDDPYSNVNCTKILQGDPEEIQKVKLEILTVQFKKRPRWTPHDYINMTRDCASFIRTRKYIVEPLTKEEVGFPIAYSIVVHHKIEMLDRLLRAIYMPQNFYCIHVDRKAEESFLAAVQGIASCFDNVFVASQLESVVYASWTRVKADLNCMKDLYRMNANWKYLINLCGMDFPIKTNLEIVRKLKCSTGENNLETEKMPPNKEERWKKRYAVVDGKLTNTGIVKAPPPLKTPLFSGSAYFVVTREYVGYVLENENIQKLMEWAQDTYSPDEFLWATIQRIPEVPGSFPSSNKYDLSDMNAIARFVKWQYFEGDVSNGAPYPPCSGVHVRSVCVFGAGDLSWMLRQHHLFANKFDMDVDPFAIQCLDEHLRRKALENLEH

SEQ ID NO: 9 - protein, 392 aa, Artificial
  Mutated human protein.
KPEFVSVRHLELAGENPSSDINCTKVLQGDVNEIQKVKLEILTVKFKKRPRWTPDDYINMTSDCSSFIKRRKYIVEPLSKEEAEFPIAYSIVVHHKIEMLDRLLRAIYMPQNFYCIHVDTKSEDSYLAAVMGIASCFSNVFVASRLESVVYASWSRVQADLNCMKDLYAMSANWKYLINLSGMDFPIKTNLEIVRKLKLLMGENNLETERMPSHKEERWKKRYEVVNGKLTNTGTVKMLPPLETPLFSGSAYFVVSREYVGYVLQNEKIQKLMEWAQDTYSPDEYLWATIQRIPEVPGSLPASHKYDLSDMQAVARFVKWQYFEGDVSKGAPYPPCDGVHVRSVCIFGAGDLNWMLRKHHLFANKFDVDVDLFAIQCLDEHLRHKALETLKH

SEQ ID NO: 10 - protein, 428 aa, Artificial
  Recombinant fusion protein.
KETAAAKFERQHMDSPPPSGLVPRGSAGSGTIDDDDKPEFVSVRHLELAGENPSSDINCTKVLQGDVNEIQKVKLEILTVKFKKRPRWTPDDYINMTSDCSSFIKRRKYIVEPLSKEEAEFPIAYSIVVHHKIEMLDRLLRAIYMPQNFYCIHVDTKSEDSYLAAVMGIASCFSNVFVASRLESVVYASWSRVQADLNCMKDLYAMSANWKYLINLSGMDFPIKTNLEIVRKLKLLMGENNLETERMPSHKEERWKKRYEVVNGKLTNTGTVKMLPPLETPLFSGSAYFVVSREYVGYVLQNEKIQKLMEWAQDTYSPDEYLWATIQRIPEVPGSLPASHKYDLSDMQAVARFVKWQYFEGDVSKGAPYPPCDGVHVRSVCIFGAGDLNWMLRKHHLFANKFDVDVDLFAIQCLDEHLRHKALETLKH

SEQ ID NO: 11 - protein, 395 aa, Artificial
  Mutated mouse protein.
IHQKPEFFSVRHLELAGDDPYSNVNCTKILQGDPEEIQKVKLEILTVQFKKRPRWTPHDYINMTRDCASFIRTRKYIVEPLTKEEVGFPIAYSIVVHHKIEMLDRLLRAIYMPQNFYCIHVDRKAEESFLAAVQGIASCFDNVFVASQLESVVYASWTRVKADLNCMKDLYRMNANWKYLINLSGMDFPIKTNLEIVRKLKCSTGENNLETEKMPPNKEERWKKRYAVVDGKLTNTGIVKAPPPLKTPLFSGSAYFVVTREYVGYVLENENIQKLMEWAQDTYSPDEFLWATIQRIPEVPGSFPSSNKYDLSDMNAIARFVKWQYFEGDVSNGAPYPPCSGVHVRSVCVFGAGDLSWMLRQHHLFANKFDMDVDPFAIQCLDEHLRRKALENLEH

SEQ ID NO: 12 - protein, 413 aa, Artificial
  Mutated mouse protein
DTKDDDDKLAAANSSIDLIHQKPEFFSVRHLELAGDDPYSNVNCTKILQGDPEEIQKVKLEILTVQFKKRPRWTPHDYINMTRDCASFIRTRKYIVEPLTKEEVGFPIAYSIVVHHKIEMLDRLLRAIYMPQNFYCIHVDRKAEESFLAAVQGIASCFDNVFVASQLESVVYASWTRVKADLNCMKDLYRMNANWKYLINLSGMDFPIKTNLEIVRKLKCSTGENNLETEKMPPNKEERWKKRYAVVDGKLTNTGIVKAPPPLKTPLFSGSAYFVVTREYVGYVLENENIQKLMEWAQDTYSPDEFLWATIQRIPEVPGSFPSSNKYDLSDMNAIARFVKWQYFEGDVSNGAPYPPCSGVHVRSVCVFGAGDLSWMLRQHHLFANKFDMDVDPFAIQCLDEHLRRKALENLEH

SEQ ID NO: 13 - DNA, 1176 bp, Artificial
  Nucleic acid encoding mutant human peptide.
aagcctgaatttgtaagtgtcagacacttggagcttgctggggagaatcctagtagtgatattaattgcaccaaagttttacagggtgatgtaaatgaaatccaaaaggtaaagcttgagatcctaacagtgaaatttaaaaagcgccctcggtggacacctgacgactatataaacatgaccagtgactgttcttctttcatcaagagacgcaaatatattgtagaaccccttagtaaagaagaggcggagtttccaatagcatattctatagtggttcatcacaagattgaaatgcttgacaggctgctgagggccatctatatgcctcagaatttctattgcattcatgtggacacaaaatccgaggattcctatttagctgcagtgatgggcatcgcttcctgttttagtaatgtctttgtggccagccgattggagagtgtggtttatgcatcgtggagccgggttcaggctgacctcaactgcatgaaggatctctatgcaatgagtgcaaactggaagtacttgataaatctttctggtatggattttcccattaaaaccaacctagaaattgtcaggaagctcaagttgttaatgggagaaaacaacctggaaacggagaggatgccatcccataaagaagaaaggtggaagaagcggtatgaggtcgttaatggaaagctgacaaacacagggactgtcaaaatgcttcctccactcgaaacacctctcttttctggcagtgcctacttcgtggtcagtagggagtatgtggggtatgtactacagaatgaaaaaatccaaaagttgatggagtgggcacaagacacatacagccctgatgagtatctctgggccaccatccaaaggattcctgaagtcccgggctcactccctgccagccataagtatgatctgtctgacatgcaagcagttgccaggtttgtcaagtggcagtactttgagggtgatgtttccaagggtgctccctacccgccctgcgatggagtccatgtgcgctcagtgtgcattttcggagctggtgacttgaactggatgctgcgcaaacaccacttgtttgccaataagtttgacgtggatgttgacctctttgccatccagtgtttggatgagcatttgaggcacaaagctttggagacattaaaacac

SEQ ID NO: 14 - DNA, 1284 bp, Artificial
  Nucleic acid encoding mutant human peptide.
aaagaaaccgctgctgcgaaatttgaacgccagcacatggactcgccaccgccttctggtctggtcccccggggcagcgcaggttctggtacgattgatgacgacgacaagcctgaatttgtaagtgtcagacacttggagcttgctggggagaatcctagtagtgatattaattgcaccaaagttttacagggtgatgtaaatgaaatccaaaaggtaaagcttgagatcctaacagtgaaatttaaaaagcgccctcggtggacacctgacgactatataaacatgaccagtgactgttcttctttcatcaagagacgcaaatatattgtagaaccccttagtaaagaagaggcggagtttccaatagcatattctatagtggttcatcacaagattgaaatgcttgacaggctgctgagggccatctatatgcctcagaatttctattgcattcatgtggacacaaaatccgaggattcctatttagctgcagtgatgggcatcgcttcctgttttagtaatgtctttgtggccagccgattggagagtgtggtttatgcatcgtggagccgggttcaggctgacctcaactgcatgaaggatctctatgcaatgagtgcaaactggaagtacttgataaatctttctggtatggattttcccattaaaaccaacctagaaattgtcaggaagctcaagttgttaatgggagaaaacaacctggaaacggagaggatgccatcccataaagaagaaaggtggaagaagcggtatgaggtcgttaatggaaagctgacaaacacagggactgtcaaaatgcttcctccactcgaaacacctctcttttctggcagtgcctacttcgtggtcagtagggagtatgtggggtatgtactacagaatgaaaaaatccaaaagttgatggagtgggcacaagacacatacagccctgatgagtatctctgggccaccatccaaaggattcctgaagtcccgggctcactccctgccagccataagtatgatctgtctgacatgcaagcagttgccaggtttgtcaagtggcagtactttgagggtgatgtttccaagggtgctccctacccgccctgcgatggagtccatgtgcgctcagtgtgcattttcggagctggtgacttgaactggatgctgcgcaaacaccacttgtttgccaataagtttgacgtggatgttgacctctttgccatccagtgtttggatgagcatttgaggcacaaagctttggagacattaaaacac

SEQ ID NO: 15 - DNA, 1188 bp, Artificial
  Nucleic acid encoding mutant mouse peptide.
attcatcagaagcctgaattttttagtgtcagacacttggagctggctggagatgatccttacagcaatgttaattgcaccaagattttacagggtgacccagaagaaatccagaaggtgaagcttgagatactaacagtgcaattcaagaagcgcccgaggtggacaccccatgactacataaacatgacccgtgactgtgcctctttcatcaggacacgcaaatatattgtggagccccttactaaagaagaggtaggctttccaattgcatattccattgtggttcatcataagattgaaatgcttgacaggctcctaagggccatctatatgcctcagaatttctactgcattcacgtggacagaaaagcagaggaatcctttttagccgcggtgcagggcattgcatcctgctttgataatgtctttgtggccagccagttggagagtgttgtttatgcgtcctggactcgggttaaagcagacctcaactgcatgaaggacctgtacagaatgaatgcaaactggaagtacttgatcaatctctctggtatggatttccctattaaaaccaacctggaaattgtcaggaagctcaagtgctccacaggggaaaacaacctggaaactgagaagatgcctcccaacaaggaagaaagatggaaaaaaagatacgccgttgtcgatgggaagctgaccaacactgggatagtcaaagcaccgcccccactcaaaactcctctcttttcaggcagtgcctacttcgtggtcactagggaatatgtaggctacgtgctggaaaatgaaaatattcaaaagttgatggaatgggcacaggacacatacagcccagatgagttcctctgggccaccatccaaaggatcccagaagtccctggttctttcccctcaagcaacaagtatgacttgtcagacatgaatgccattgctaggtttgtcaagtggcagtacttcgaaggcgatgtttccaatggtgccccttatccaccgtgcagtggagtccacgtgcgctctgtgtgcgtcttcggagctggtgacttgagctggatgctgcgccaacaccacctttttgccaataagtttgacatggatgtcgacccctttgccatccagtgtttggatgagcatctgaggcgtaaagccctggagaacttagaacactaa

SEQ ID NO: 16 - DNA, 1242 bp, Artificial
  Nucleic acid encoding mutant mouse peptide.
gactacaaagacgatgacgacaagcttgcggccgcgaattcatcgatagatctaattcatcagaagcctgaattttttagtgtcagacacttggagctggctggagatgatccttacagcaatgttaattgcaccaagattttacagggtgacccagaagaaatccagaaggtgaagcttgagatactaacagtgcaattcaagaagcgcccgaggtggacaccccatgactacataaacatgacccgtgactgtgcctctttcatcaggacacgcaaatatattgtggagccccttactaaagaagaggtaggctttccaattgcatattccattgtggttcatcataagattgaaatgcttgacaggctcctaagggccatctatatgcctcagaatttctactgcattcacgtggacagaaaagcagaggaatcctttttagccgcggtgcagggcattgcatcctgctttgataatgtctttgtggccagccagttggagagtgttgtttatgcgtcctggactcgggttaaagcagacctcaactgcatgaaggacctgtacagaatgaatgcaaactggaagtacttgatcaatctctctggtatggatttccctattaaaaccaacctggaaattgtcaggaagctcaagtgctccacaggggaaaacaacctggaaactgagaagatgcctcccaacaaggaagaaagatggaaaaaaagatacgccgttgtcgatgggaagctgaccaacactgggatagtcaaagcaccgcccccactcaaaactcctctcttttcaggcagtgcctacttcgtggtcactagggaatatgtaggctacgtgctggaaaatgaaaatattcaaaagttgatggaatgggcacaggacacatacagcccagatgagttcctctgggccaccatccaaaggatcccagaagtccctggttctttcccctcaagcaacaagtatgacttgtcagacatgaatgccattgctaggtttgtcaagtggcagtacttcgaaggcgatgtttccaatggtgccccttatccaccgtgcagtggagtccacgtgcgctctgtgtgcgtcttcggagctggtgacttgagctggatgctgcgccaacaccacctttttgccaataagtttgacatggatgtcgacccctttgccatccagtgtttggatgagcatctgaggcgtaaagccctggagaacttagaacactaa

SEQ ID NO: 17 - protein, 392 aa, Artificial
  Nucleic acid encoding mutant human peptide.
KPEFVSVRHLELAGENPSSDINCTKVLQGDVNEIQKVKLEILTVKFKKRPRWTPDDYINMTSDCSSFIKRRKYIVEPLSKEEAEFPIAYSIVVHHKIEMLDRLLRAIYMPQNFYCIHVDTKSEDSYLAAVMGIASCFSNVFVASRLESVVYASWSRVQADLNCMKDLYAMSANWKYLINLAGMDFPIKTNLEIVRKLKLLMGENNLETERMPSHKEERWKKRYEVVNGKLTNTGTVKMLPPLETPLFSGSAYFVVSREYVGYVLQNEKIQKLMEWAQDTYSPDEYLWATIQRIPEVPGSLPASHKYDLSDMQAVARFVKWQYFEGDVSKGAPYPPCDGVHVRSVCIFGAGDLNWMLRKHHLFANKFDVDVDLFAIQCLDEHLRHKALETLKH

SEQ ID NO: 18 - protein, 428 aa, Artificial
  Nucleic acid encoding mutant human peptide.
KETAAAKFERQHMDSPPPSGLVPRGSAGSGTIDDDDKPEFVSVRHLELAGENPSSDINCTKVLQGDVNEIQKVKLEILTVKFKKRPRWTPDDYINMTSDCSSFIKRRKYIVEPLSKEEAEFPIAYSIVVHHKIEMLDRLLRAIYMPQNFYCIHVDTKSEDSYLAAVMGIASCFSNVFVASRLESVVYASWSRVQADLNCMKDLYAMSANWKYLINLAGMDFPIKTNLEIVRKLKLLMGENNLETERMPSHKEERWKKRYEVVNGKLTNTGTVKMLPPLETPLFSGSAYFVVSREYVGYVLQNEKIQKLMEWAQDTYSPDEYLWATIQRIPEVPGSLPASHKYDLSDMQAVARFVKWQYFEGDVSKGAPYPPCDGVHVRSVCIFGAGDLNWMLRKHHLFANKFDVDVDLFAIQCLDEHLRHKALETLKH

SEQ ID NO: 19 - protein, 395 aa, Artificial
  Nucleic acid encoding mutant mouse peptide.
IHQKPEFFSVRHLELAGDDPYSNVNCTKILQGDPEEIQKVKLEILTVQFKKRPRWTPHDYINMTRDCASFIRTRKYIVEPLTKEEVGFPIAYSIVVHHKIEMLDRLLRAIYMPQNFYCIHVDRKAEESFLAAVQGIASCFDNVFVASQLESVVYASWTRVKADLNCMKDLYRMNANWKYLINLAGMDFPIKTNLEIVRKLKCSTGENNLETEKMPPNKEERWKKRYAVVDGKLTNTGIVKAPPPLKTPLFSGSAYFVVTREYVGYVLENENIQKLMEWAQDTYSPDEFLWATIQRIPEVPGSFPSSNKYDLSDMNAIARFVKWQYFEGDVSNGAPYPPCSGVHVRSVCVFGAGDLSWMLRQHHLFANKFDMDVDPFAIQCLDEHLRRKALENLEH

SEQ ID NO: 20 - protein, 413 aa, Artificial
  Nucleic acid encoding mutant mouse peptide.
DTKDDDDKLAAANSSIDLIHQKPEFFSVRHLELAGDDPYSNVNCTKILQGDPEEIQKVKLEILTVQFKKRPRWTPHDYINMTRDCASFIRTRKYIVEPLTKEEVGFPIAYSIVVHHKIEMLDRLLRAIYMPQNFYCIHVDRKAEESFLAAVQGIASCFDNVFVASQLESVVYASWTRVKADLNCMKDLYRMNANWKYLINLAGMDFPIKTNLEIVRKLKCSTGENNLETEKMPPNKEERWKKRYAVVDGKLTNTGIVKAPPPLKTPLFSGSAYFVVTREYVGYVLENENIQKLMEWAQDTYSPDEFLWATIQRIPEVPGSFPSSNKYDLSDMNAIARFVKWQYFEGDVSNGAPYPPCSGVHVRSVCVFGAGDLSWMLRQHHLFANKFDMDVDPFAIQCLDEHLRRKALENLEH

SEQ ID NO: 21 - DNA, 1176 bp, Artificial
  Nucleic acid encoding mutant human peptide.
aagcctgaatttgtaagtgtcagacacttggagcttgctggggagaatcctagtagtgatattaattgcaccaaagttttacagggtgatgtaaatgaaatccaaaaggtaaagcttgagatcctaacagtgaaatttaaaaagcgccctcggtggacacctgacgactatataaacatgaccagtgactgttcttctttcatcaagagacgcaaatatattgtagaaccccttagtaaagaagaggcggagtttccaatagcatattctatagtggttcatcacaagattgaaatgcttgacaggctgctgagggccatctatatgcctcagaatttctattgcattcatgtggacacaaaatccgaggattcctatttagctgcagtgatgggcatcgcttcctgttttagtaatgtctttgtggccagccgattggagagtgtggtttatgcatcgtggagccgggttcaggctgacctcaactgcatgaaggatctctatgcaatgagtgcaaactggaagtacttgataaatcttgctggtatggattttcccattaaaaccaacctagaaattgtcaggaagctcaagttgttaatgggagaaaacaacctggaaacggagaggatgccatcccataaagaagaaaggtggaagaagcggtatgaggtcgttaatggaaagctgacaaacacagggactgtcaaaatgcttcctccactcgaaacacctctcttttctggcagtgcctacttcgtggtcagtagggagtatgtggggtatgtactacagaatgaaaaaatccaaaagttgatggagtgggcacaagacacatacagccctgatgagtatctctgggccaccatccaaaggattcctgaagtcccgggctcactccctgccagccataagtatgatctgtctgacatgcaagcagttgccaggtttgtcaagtggcagtactttgagggtgatgtttccaagggtgctccctacccgccctgcgatggagtccatgtgcgctcagtgtgcattttcggagctggtgacttgaactggatgctgcgcaaacaccacttgtttgccaataagtttgacgtggatgttgacctctttgccatccagtgtttggatgagcatttgaggcacaaagctttggagacattaaaacac

SEQ ID NO: 22 - DNA, 1284 bp, Artificial
  Nucleic acid encoding mutant human peptide.
aaagaaaccgctgctgcgaaatttgaacgccagcacatggactcgccaccgccttctggtctggtcccccggggcagcgcaggttctggtacgattgatgacgacgacaagcctgaatttgtaagtgtcagacacttggagcttgctggggagaatcctagtagtgatattaattgcaccaaagttttacagggtgatgtaaatgaaatccaaaaggtaaagcttgagatcctaacagtgaaatttaaaaagcgccctcggtggacacctgacgactatataaacatgaccagtgactgttcttctttcatcaagagacgcaaatatattgtagaaccccttagtaaagaagaggcggagtttccaatagcatattctatagtggttcatcacaagattgaaatgcttgacaggctgctgagggccatctatatgcctcagaatttctattgcattcatgtggacacaaaatccgaggattcctatttagctgcagtgatgggcatcgcttcctgttttagtaatgtctttgtggccagccgattggagagtgtggtttatgcatcgtggagccgggttcaggctgacctcaactgcatgaaggatctctatgcaatgagtgcaaactggaagtacttgataaatcttgctggtatggattttcccattaaaaccaacctagaaattgtcaggaagctcaagttgttaatgggagaaaacaacctggaaacggagaggatgccatcccataaagaagaaaggtggaagaagcggtatgaggtcgttaatggaaagctgacaaacacagggactgtcaaaatgcttcctccactcgaaacacctctcttttctggcagtgcctacttcgtggtcagtagggagtatgtggggtatgtactacagaatgaaaaaatccaaaagttgatggagtgggcacaagacacatacagccctgatgagtatctctgggccaccatccaaaggattcctgaagtcccgggctcactccctgccagccataagtatgatctgtctgacatgcaagcagttgccaggtttgtcaagtggcagtactttgagggtgatgtttccaagggtgctccctacccgccctgcgatggagtccatgtgcgctcagtgtgcattttcggagctggtgacttgaactggatgctgcgcaaacaccacttgtttgccaataagtttgacgtggatgttgacctctttgccatccagtgtttggatgagcatttgaggcacaaagctttggagacattaaaacac

SEQ ID NO: 23 - DNA, 1188 bp, Artificial
  Nucleic acid encoding mutant mouse peptide.
attcatcagaagcctgaattttttagtgtcagacacttggagctggctggagatgatccttacagcaatgttaattgcaccaagattttacagggtgacccagaagaaatccagaaggtgaagcttgagatactaacagtgcaattcaagaagcgcccgaggtggacaccccatgactacataaacatgacccgtgactgtgcctctttcatcaggacacgcaaatatattgtggagccccttactaaagaagaggtaggctttccaattgcatattccattgtggttcatcataagattgaaatgcttgacaggctcctaagggccatctatatgcctcagaatttctactgcattcacgtggacagaaaagcagaggaatcctttttagccgcggtgcagggcattgcatcctgctttgataatgtctttgtggccagccagttggagagtgttgtttatgcgtcctggactcgggttaaagcagacctcaactgcatgaaggacctgtacagaatgaatgcaaactggaagtacttgatcaatctcgctggtatggatttccctattaaaaccaacctggaaattgtcaggaagctcaagtgctccacaggggaaaacaacctggaaactgagaagatgcctcccaacaaggaagaaagatggaaaaaaagatacgccgttgtcgatgggaagctgaccaacactgggatagtcaaagcaccgcccccactcaaaactcctctcttttcaggcagtgcctacttcgtggtcactagggaatatgtaggctacgtgctggaaaatgaaaatattcaaaagttgatggaatgggcacaggacacatacagcccagatgagttcctctgggccaccatccaaaggatcccagaagtccctggttctttcccctcaagcaacaagtatgacttgtcagacatgaatgccattgctaggtttgtcaagtggcagtacttcgaaggcgatgtttccaatggtgccccttatccaccgtgcagtggagtccacgtgcgctctgtgtgcgtcttcggagctggtgacttgagctggatgctgcgccaacaccacctttttgccaataagtttgacatggatgtcgacccctttgccatccagtgtttggatgagcatctgaggcgtaaagccctggagaacttagaacactaa

SEQ ID NO: 24 - DNA, 1242 bp, Artificial
  Nucleic acid encoding mutant mouse peptide.
gactacaaagacgatgacgacaagcttgcggccgcgaattcatcgatagatctaattcatcagaagcctgaattttttagtgtcagacacttggagctggctggagatgatccttacagcaatgttaattgcaccaagattttacagggtgacccagaagaaatccagaaggtgaagcttgagatactaacagtgcaattcaagaagcgcccgaggtggacaccccatgactacataaacatgacccgtgactgtgcctctttcatcaggacacgcaaatatattgtggagccccttactaaagaagaggtaggctttccaattgcatattccattgtggttcatcataagattgaaatgcttgacaggctcctaagggccatctatatgcctcagaatttctactgcattcacgtggacagaaaagcagaggaatcctttttagccgcggtgcagggcattgcatcctgctttgataatgtctttgtggccagccagttggagagtgttgtttatgcgtcctggactcgggttaaagcagacctcaactgcatgaaggacctgtacagaatgaatgcaaactggaagtacttgatcaatctcgctggtatggatttccctattaaaaccaacctggaaattgtcaggaagctcaagtgctccacaggggaaaacaacctggaaactgagaagatgcctcccaacaaggaagaaagatggaaaaaaagatacgccgttgtcgatgggaagctgaccaacactgggatagtcaaagcaccgcccccactcaaaactcctctcttttcaggcagtgcctacttcgtggtcactagggaatatgtaggctacgtgctggaaaatgaaaatattcaaaagttgatggaatgggcacaggacacatacagcccagatgagttcctctgggccaccatccaaaggatcccagaagtccctggttctttcccctcaagcaacaagtatgacttgtcagacatgaatgccattgctaggtttgtcaagtggcagtacttcgaaggcgatgtttccaatggtgccccttatccaccgtgcagtggagtccacgtgcgctctgtgtgcgtcttcggagctggtgacttgagctggatgctgcgccaacaccacctttttgccaataagtttgacatggatgtcgacccctttgccatccagtgtttggatgagcatctgaggcgtaaagccctggagaacttagaacactaa

SEQ ID NO: 25 - protein, 13 aa, Artificial
  Recombinantly exprssed mutant peptide.
WXYXINXCGXDFP

SEQ ID NO: 26 - DNA, 33 bp, Artificial
  Synthetic oligonucleotide primer.
tacttgatcaatctctctggtatggatttccct

SEQ ID NO: 27 - DNA, 33 bp, Artificial
  Synthetic oligonucleotide primer.
tacttgatcaatctcactggtatggatttccct

SEQ ID NO: 28 - DNA, 33 bp, Artificial
  Synthetic oligonucleotide primer.
tacttgatcaatctcgctggtatggatttccct

SEQ ID NO: 29 - DNA, 1284 bp, Mus musculus
aaagaaaccgctgctgcgaaatttgaacgccagcacatggactcgccaccgccttctggtctggtcccccggggcagcgcaggttctggtacgattgatgacgacgacaagcctgaatttgtaagtgtcagacacttggagcttgctggggagaatcctagtagtgatattaattgcaccaaagttttacagggtgatgtaaatgaaatccaaaaggtaaagcttgagatcctaacagtgaaatttaaaaagcgccctcggtggacacctgacgactatataaacatgaccagtgactgttcttctttcatcaagagacgcaaatatattgtagaaccccttagtaaagaagaggcggagtttccaatagcatattctatagtggttcatcacaagattgaaatgcttgacaggctgctgagggccatctatatgcctcagaatttctattgcattcatgtggacacaaaatccgaggattcctatttagctgcagtgatgggcatcgcttcctgttttagtaatgtctttgtggccagccgattggagagtgtggtttatgcatcgtggagccgggttcaggctgacctcaactgcatgaaggatctctatgcaatgagtgcaaactggaagtacttgataaatctttgtggtatggattttcccattaaaaccaacctagaaattgtcaggaagctcaagttgttaatgggagaaaacaacctggaaacggagaggatgccatcccataaagaagaaaggtggaagaagcggtatgaggtcgttaatggaaagctgacaaacacagggactgtcaaaatgcttcctccactcgaaacacctctcttttctggcagtgcctacttcgtggtcagtagggagtatgtggggtatgtactacagaatgaaaaaatccaaaagttgatggagtgggcacaagacacatacagccctgatgagtatctctgggccaccatccaaaggattcctgaagtcccgggctcactccctgccagccataagtatgatctgtctgacatgcaagcagttgccaggtttgtcaagtggcagtactttgagggtgatgtttccaagggtgctccctacccgccctgcgatggagtccatgtgcgctcagtgtgcattttcggagctggtgacttgaactggatgctgcgcaaacaccacttgtttgccaataagtttgacgtggatgttgacctctttgccatccagtgtttggatgagcatttgaggcacaaagctttggagacattaaaacac

SEQ ID NO: 30 - DNA, 1242 bp, Mus musculus
gactacaaagacgatgacgacaagcttgcggccgcgaattcatcgatagatctaattcatcagaagcctgaattttttagtgtcagacacttggagctggctggagatgatccttacagcaatgttaattgcaccaagattttacagggtgacccagaagaaatccagaaggtgaagcttgagatactaacagtgcaattcaagaagcgcccgaggtggacaccccatgactacataaacatgacccgtgactgtgcctctttcatcaggacacgcaaatatattgtggagccccttactaaagaagaggtaggctttccaattgcatattccattgtggttcatcataagattgaaatgcttgacaggctcctaagggccatctatatgcctcagaatttctactgcattcacgtggacagaaaagcagaggaatcctttttagccgcggtgcagggcattgcatcctgctttgataatgtctttgtggccagccagttggagagtgttgtttatgcgtcctggactcgggttaaagcagacctcaactgcatgaaggacctgtacagaatgaatgcaaactggaagtacttgatcaatctctgtggtatggatttccctattaaaaccaacctggaaattgtcaggaagctcaagtgctccacaggggaaaacaacctggaaactgagaagatgcctcccaacaaggaagaaagatggaaaaaaagatacgccgttgtcgatgggaagctgaccaacactgggatagtcaaagcaccgcccccactcaaaactcctctcttttcaggcagtgcctacttcgtggtcactagggaatatgtaggctacgtgctggaaaatgaaaatattcaaaagttgatggaatgggcacaggacacatacagcccagatgagttcctctgggccaccatccaaaggatcccagaagtccctggttctttcccctcaagcaacaagtatgacttgtcagacatgaatgccattgctaggtttgtcaagtggcagtacttcgaaggcgatgtttccaatggtgccccttatccaccgtgcagtggagtccacgtgcgctctgtgtgcgtcttcggagctggtgacttgagctggatgctgcgccaacaccacctttttgccaataagtttgacatggatgtcgacccctttgccatccagtgtttggatgagcatctgaggcgtaaagccctggagaacttagaacactaa